(19) United States
(12) Patent Application Publication
(10) Pub. No.: US 2014/0275091 A1
(43) Pub. Date: Sep. 18, 2014

(54) Ocular formulations comprising geranylgeranylacetone derivatives for intraocular delivery

(71) Applicant: COYOTE PHARMACEUTICALS, INC., Menlo Park, CA (US)

(72) Inventors: Hiroaki Serizawa, Menlo Park, CA (US); Tilmann M. Brotz, Menlo Park, CA (US)

(21) Appl. No.: 13/815,870

(22) Filed: Mar. 15, 2013

(51) Int. Cl. A61K 9/00

(52) U.S. Cl. CPC A61K 9/0048
USPC 514/237.8; 514/480; 514/367; 514/352; 514/315

(57) ABSTRACT

Provided herein is a pharmaceutical formulation comprising a GGA derivative in the form of an eye drop. Also provided herein are methods of treating neural diseases or disorders by administering such pharmaceutical formulations.

### FIELD OF THE INVENTION

This invention relates to ocular formulations of GGA derivatives and methods of using them.

### STATE OF THE ART

It is difficult at best for an agent to penetrate into the eye and be delivered intraocularly. There is a need for delivering therapeutic agents into the eye, for example, for therapeutic purposes.

### SUMMARY OF THE INVENTION

It is contemplated that GGA derivatives can effectively penetrate into ocular tissue when administered on the ocular surface, or administered ocularly by topical delivery. As used herein “ocular” delivery refers to intraocular and/or topical delivery. In some embodiments, the GGA derivative is delivered into the eye or preferably into the retina of the subject. In certain aspects, this invention relates to pharmaceutical uses of GGA derivatives, pharmaceutical compositions of GGA derivatives, and methods of using such compounds and pharmaceutical compositions. In some embodiments, based on dose-adjusted AUC, topical ocular administration is 350 to 3700-times more efficient in delivering a GGA derivative to the eye ball or retina than oral administration.

In another aspect provide herein are compounds wherein GGA or a derivative thereof is conjugated to an anti-cancer agent. In one embodiment, the conjugate is of formula:

[chemical structure]

wherein R¹-R⁵, m, and n are defined as in Formula (II) herein, L¹⁰ is a bond or a linker joining the isoprenyl portion to the Drug, and the Drug is preferably an antibiotic or a glaucoma drug, or is an anticancer agent, or is an antiviral agent. In certain preferred embodiments, the linker is a bond, methylene, or carbonyl. In certain other preferred embodiments, the linker joins the isoprenyl portion to a carbonyl moiety, or an oxygen, nitrogen, or sulfur atom of the drug. In yet another preferred embodiment, R¹-R⁵ are methyl, and m and n are 1. Such conjugates are formulated and administered in accordance with this invention.

In some embodiments, the GGA derivative is formulated as a thermosensitive gel. Thus formulated, a precursor sol is administered on the ocular surface where at an increased temperature, the sol undergoes a sol to gel transition. In some preferred embodiments, such gels comprise Polaxamers® as excipients. In some embodiments, the eye drop formulation forms a colored film once it contacts the ocular surface. Such a coloration allows an attending physician to determine the extent of the eye drop formulation retained on the ocular surface, and not spilled away from it, after delivery.

According to an aspect of this invention, a method is provided for inhibiting optic nerve damage in a patient at risk of such damage which method comprises applying a therapeutically effective amount of a composition comprising 0.0001 wt %-20 wt % of a GGA derivative to or into an ocular surface of said patient in an amount sufficient to increase intraocular levels of HSP 70, thereby inhibiting the optic nerve damage. In some preferred embodiments, the composition comprises 0.1 wt % to 10 wt % of a GGA derivative. In other preferred embodiments, the composition comprises 3 wt % to 6 wt % of a GGA derivative. In one embodiment, the invention provides a method for delivering unexpectedly high intraocular levels of a GGA derivative by administering a GGA derivative to an ocular surface of said patient.

According to another aspect of this invention, a method is provided for delivering a GGA derivative to the brain and/or the spinal chord of a patient, which method comprises applying a composition comprising the GGA derivative to an ocular surface or into the intraocular tissue of said patient in an amount sufficient to introduce an effective amount of GGA derivative into the brain and/or the spinal chord. Without being bound by theory, it is contemplated that after administration of the GGA derivative to an ocular surface or into intraocular tissue, the GGA derivative passes through the blood-brain barrier to deliver an effective amount of GGA to the brain and/or the spinal chord. As used herein, an effective amount refers to a therapeutically effective amount or to a an amount effectively measured in the brain and/or the spinal chord.

According to yet another aspect of this invention, a method is provided for increasing HSP70 levels in ocular tissue comprising administering topically on the ocular surface an effective amount of a GGA derivative.

In other embodiments of this invention, the method further includes providing an intraocular concentration of the GGA derivative. In some embodiments of this invention, the intraocular levels of HSP 70 may be increased by at least 10%. In other embodiments of this invention, the optic nerve damage derives from or is related to glaucoma, macular degeneration, exposure to UV light, trauma, stroke, optic neuritis, ischemia, infection, compression from a tumor, compression from an aneurysm or Leber's hereditary optic neuropathy.

According to yet another aspect of this invention, a pharmaceutical composition is provided, where the pharmaceutical composition is suitable for parenteral administration through the ocular surface of a patient, wherein the pharmaceutical composition comprises a GGA derivative and at least one excipient for introducing the GGA derivative into the eye of a subject. In some embodiments of this invention, the pharmaceutical composition is suitable for parenteral administration through the ocular surface of a patient via a jetting device.

According to still another aspect of this invention, a pharmaceutical composition suitable for topical administration to a patient is provided, where the pharmaceutical composition comprises less than 0.05 wt % of a GGA derivative and at least one excipient for introducing the GGA derivative into the eye of a subject, provided that the composition does not include an egg-based excipient, such as, for example, an egg-based phospholipid. Based on the surprising discoveries discussed herein, It is contemplated that even such small concentrations are suitable for administering a therapeutically effective amount of a GGA derivative, preferably into the eye and also to the brain, and/or the spinal chord.

Thus, in one embodiment, the invention provides pharmaceutical compositions suitable for topical administration that despite having low concentrations of a GGA derivative, deliver an effective concentration of a GGA derivative to a patient via the topical route. In certain preferred embodiments, the pharmaceutical composition comprises less than 0.5 wt % of a GGA derivative. In other preferred embodiments, the pharmaceutical composition comprises less than 0.05 wt % of a GGA derivative. In certain embodiments, the excipient for introducing the GGA derivative into the eye of a subject comprises a tonicity adjustment agent.

In some preferred embodiments, the GGA derivative is co-administered or administered in combination with beta-blockers and a steroid such as prostaglandin. Topical formulations, preferably ocular formulations, including GGA derivative and one or more of a beta-blocker and a steroid, and uses thereof, preferably in treating optic nerve damage, such as those relating from glaucoma, are also contemplated according to this invention.

Provided herein, in some embodiments, is a topical ocular composition comprising a GGA derivative, and at least one tonicity adjusting agent. In some embodiments, the isotonic tonicity adjusting agent is isotonic. In specific embodiments, the tonicity adjusting agent is saline, dextrose, glycerin, aqueous potassium chloride, buffer salts, propylene glycol, or mannitol. In certain specific embodiments, the tonicity adjusting agent is saline. In some embodiments provided herein, the topical ocular composition is formulated as a topical eye drop. In some embodiments, the composition comprises about 0.1-20% of a GGA derivative. In some embodiments, the composition comprises about 0.1-10%, 0.1-2%, 0.1-1%, or 0.05-1% of the GGA derivative.

In some embodiments, the topical ocular composition further comprises one or more of a surfactant, an anti-bacterial agent, a pH buffering agent, an antioxidant agent, a preservative agent, a viscosity imparting agent or a combination thereof. In further or additional embodiments, the topical ocular composition is used for the manufacture of a medicament for the treatment of an ocular or visual disorder. In some embodiments, the ocular or visual disorder is a neurodegenerative disorder. In specific embodiments, the ocular or visual disorder is glaucoma, optic nerve degeneration or age-related macular degeneration.

Also provided herein in some embodiments is a physiological supplement or medicament for ophthalmic use, in the form of eye drops, comprising a GGA derivative in a range of about 0.1-20% of a GGA derivative. In some embodiments, the composition comprises about 0.1-10%, 0.1-2%, 0.1-1%, or 0.05-1% of the GGA derivative.

Some embodiments provided herein describe a formulation for treatment of an ocular neural disease, disorder or condition, comprising a GGA derivative, and at least one carrier material for introducing the GGA derivative into the eye of a subject suffering from the ocular neural disease, disorder or condition. In some embodiments, the formulation further comprises one or more of a surfactant, an anti-bacterial agent, a pH buffering agent, an antioxidant agent, a preservative agent, or a combination thereof. In some embodiments, the carrier material comprises an ocular/ophthalmic carrier. In some embodiments, the ocular neural disease, disorder, or condition is glaucoma, optic nerve degeneration or age-related macular degeneration.

Also provided herein in some embodiments is a method of treating glaucoma, the method comprising administering to a subject in need thereof a pharmaceutical formulation comprising a GGA derivative. In further or additional embodiments, the formulation further comprises one or more of a tonicity adjusting agent, a surfactant, an anti-bacterial agent, a pH buffering agent, an antioxidant agent, a preservative agent, a viscosity imparting agent or a combination thereof. In some embodiments, the formulation comprises about 0.1-20% of a GGA derivative. In some embodiments, the composition comprises about 0.1-10%, 0.1-2%, 0.1-1%, or 0.05-1% of the GGA derivative. In some embodiments, the formulation is administered to the eye of the subject.

Some embodiments provided herein describe a method of inhibiting apoptosis of a retinal ganglion cell, the method comprising administration of a pharmaceutical formulation of a GGA derivative to the cell. In further or additional embodiments, the pharmaceutical formulation further comprises an ocular/ophthalmic carrier. In certain embodiments, the retinal ganglion cell is present in an individual. In some embodiments, the individual is in need of glaucoma therapy. In some embodiments, the pharmaceutical formulation is administered to the subject by an eye drop.

Provided herein in certain embodiments, is an eye drop for the treatment of an ocular neural disease, disorder or condition through topical application of said eye drop to the eye of a subject suffering from said disease, disorder or condition, comprising a therapeutically effective amount of a GGA derivative and a solvent for said compound which is suitable for topical application to the eye of the subject. In yet other embodiments, various bacterial and viral disorders, and cancers of the eye, brain, and spinal chord, and the nerves in the brain, eye, and the spinal chord are treated in accordance with this invention. In some embodiments, the disorder is glaucoma. In another embodiment, the disorder is herpes.

### BRIEF DESCRIPTION OF THE FIGURE

FIG. 1 illustrates the visual appearance of 0.005-5% GGA eye drop formulations.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### CERTAIN DEFINITIONS

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations. Each numerical parameter should at least be construed in light of the number of reported significant digits and by applying ordinary rounding techniques.

As used herein, Cₘ-Cₙ, such as C₁-C₁₀, C₁-C₆, or C₁-C₄ when used before a group refers to that group containing m to n carbon atoms.

The term “about” when used before a numerical designation, e.g., temperature, time, amount, and concentration, including range, indicates approximations which may vary by (+) or (−) 10%, 5% or 1%.

The term “alkoxy” refers to —O-alkyl.

The term “alkyl” refers to monovalent saturated aliphatic hydrocarbyl groups having from 1 to 10 carbon atoms (i.e., C₁-C₁₀ alkyl) or 1 to 6 carbon atoms (i.e., C₁-C₆ alkyl), or 1 to 4 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). In some embodiments, the term “alkyl” refers to substituted or unsubstituted, straight chain or branched alkyl groups with C₁-C₁₂, C₁-C₆ and preferably C₁-C₄ carbon atoms.

The term “alkenyl” refers to monovalent aliphatic hydrocarbyl groups having from 2 to 25 carbon atoms or 2 to 6 carbon atoms and 1 or more, preferably 1, carbon carbon double bond. Examples of alkenyl include vinyl, allyl, dimethyl allyl, and the like.

The term “alkynyl” refers to monovalent aliphatic hydrocarbyl groups having from 2 to 10 carbon atoms or 2 to 6 carbon atoms and 1 or more, preferably 1, carbon carbon triple bond —(C≡C)—. Examples of alkynyl include ethynyl, propargyl, dimethylpropargyl, and the like.

The term “acyl” refers to —C(O)-alkyl, where alkyl is as defined above.

The term “nitro” refers to —NO₂.

The term “aryl” refers to a monovalent, aromatic mono- or bicyclic ring having 6-10 ring carbon atoms. Examples of aryl include phenyl and naphthyl. The condensed ring may or may not be aromatic provided that the point of attachment is at an aromatic carbon atom. For example, and without limitation, the following is an aryl group:

[chemical structure]

In some embodiments, the term “aryl” refers to a 6 to 10 membered, preferably 6 membered aryl group. An aryl group may be substituted with 1-5, preferably 1-3, halo, alkyl, and/or —O-alkyl groups.

The term “—CO₂H ester” refers to an ester formed between the —CO₂H group and an alcohol, preferably an aliphatic alcohol. A preferred example included —CO₂R^{E}, wherein R^{E} is alkyl or aryl group optionally substituted with an amino group.

“Co-crystal,” or as sometimes referred to herein “co-precipitate” refers to a solid, preferably a crystalline solid, comprising GGA or a GGA derivative, and urea or thiourea, more preferably, where, the GGA or the GGA derivative reside within the urea or thiourea lattice, such as in channels formed by urea or thiourea.

“Complexed” refers to GGA or a GGA derivative bound by certain quantifiable intermolecular forces, non-limiting examples of which include hydrogen bonding and Van-Der Waals' interactions, and also by entropic effects.

The term “chiral moiety” refers to a moiety that is chiral. Such a moiety can possess one or more asymmetric centers. Preferably, the chiral moiety is enantiomerically enriched, and more preferably a single enantiomer. Non limiting examples of chiral moieties include chiral carboxylic acids, chiral amines, chiral amino acids, such as the naturally occurring amino acids, chiral alcohols including chiral steroids, and the likes.

The term “cycloalkyl” refers to a monovalent, preferably saturated, hydrocarbyl mono-, bi-, or tricyclic ring having 3-12 ring carbon atoms. While cycloalkyl, refers preferably to saturated hydrocarbyl rings, as used herein, it also includes rings containing 1-2 carbon-carbon double bonds. Nonlimiting examples of cycloalkyl include cyclopropyl, cyclobutyl, cyclopentyl, cyclohexyl, cycloheptyl, adamentyl, and the like. The condensed rings may or may not be non-aromatic hydrocarbyl rings provided that the point of attachment is at a cycloalkyl carbon atom. For example, and without limitation, the following is a cycloalkyl group:

[chemical structure]

The term “halo” refers to F, Cl, Br, and/or I.

The term “heteroaryl” refers to a monovalent, aromatic mono-, bi-, or tricyclic ring having 2-14 ring carbon atoms and 1-6 ring heteroatoms selected preferably from N, O, S, and P and oxidized forms of N, S, and P, provided that the ring contains at least 5 ring atoms. Nonlimiting examples of heteroaryl include furan, imidazole, oxadiazole, oxazole, pyridine, quinoline, and the like. The condensed rings may or may not be a heteroatom containing aromatic ring provided that the point of attachment is a heteroaryl atom. For example, and without limitation, the following is a heteroaryl group:

[chemical structure]

The term “heterocyclyl” or heterocycle refers to a non-aromatic, mono-, bi-, or tricyclic ring containing 2-10 ring carbon atoms and 1-6 ring heteroatoms selected preferably from N, O, S, and P and oxidized forms of N, S, and P, provided that the ring contains at least 3 ring atoms. While heterocyclyl preferably refers to saturated ring systems, it also includes ring systems containing 1-3 double bonds, provided that they ring is non-aromatic. Nonlimiting examples of heterocyclyl include, azalactones, oxazoline, piperidinyl, piperazinyl, pyrrolidinyl, tetrahydrofuranyl, and tetrahydropyranyl. The condensed rings may or may not contain a non-aromatic heteroatom containing ring provided that the point of attachment is a heterocyclyl group. For example, and without limitation, the following is a heterocyclyl group:

[chemical structure]

The term “hydrolyzing” refers to breaking an R^{H}—O—CO—, R^{H}—O—CS—, or an R^{H}—O—SO₂— moiety to an R^{H}—OH, preferably by adding water across the broken bond. A hydrolyzing is performed using various methods well known to the skilled artisan, non limiting examples of which include acidic and basic hydrolysis.

The term “oxo” refers to a C═O group, and to a substitution of 2 geminal hydrogen atoms with a C═O group.

The term “pharmaceutically acceptable” refers to safe and non-toxic for in vivo, preferably, human administration.

The term “pharmaceutically acceptable salt” refers to a salt that is pharmaceutically acceptable.

The term “salt” refers to an ionic compound formed between an acid and a base. When the compound provided herein contains an acidic functionality, such salts include, without limitation, alkai metal, alkaline earth metal, and ammonium salts. As used herein, ammonium salts include, salts containing protonated nitrogen bases and alkylated nitrogen bases. Exemplary, and non-limiting cations useful in pharmaceutically acceptable salts include Na, K, Rb, Cs, NH₄, Ca, Ba, imidazolium, and ammonium cations based on naturally occurring amino acids. When the compounds provided and/or utilized herein contain basic functionality, such salts include, without limitation, salts of organic acids, such as carboxylic acids and sulfonic acids, and mineral acids, such as hydrogen halides, sulfuric acid, phosphoric acid, and the likes. Exemplary and non-limiting anions useful in pharmaceutically acceptable salts include oxalate, maleate, acetate, propionate, succinate, tartrate, chloride, sulfate, bisalfate, mono-, di-, and tribasic phosphate, mesylate, tosylate, and the likes.

“Trans” in the context of GGA and GGA derivatives refer to the GGA scaffold as illustrated below:

[chemical structure]

wherein R¹-R⁵ is defined herein and q is 0-2. As shown, each double bond is in a trans or E configuration. In contrast, a cis form of GGA or a GGA derivative will contain one or more of these bonds in a cis or Z configuration.

The term “neuroprotective” refers to reduced toxicity of ocular neurons as measured, e.g., in vitro in assays where ocular neurons susceptible to degradation are protected against degradation as compared to control. Neuroprotective effects may also be evaluated in vivo by counting neurons in histology sections.

The term “neuron” or “neurons” refers to all electrically excitable cells that make up the ocular nervous system. The neurons may be cells within the body of an animal or cells cultured outside the body of an animal. The term “neuron” or “neurons” also refers to established or primary tissue culture cell lines that are derived from neural cells from a mammal or tissue culture cell lines that are made to differentiate into neurons. “Neuron” or “neurons” also refers to any of the above types of cells that have also been modified to express a particular protein either extrachromosomally or intrachromosomally. “Neuron” or “neurons” also refers to transformed neurons such as neuroblastoma cells and support cells within the brain such as glia.

The term “protein aggregates” refers to a collection of proteins that may be partially or entirely mis-folded. The protein aggregates may be soluble or insoluble and may be inside the cell or outside the cell in the space between cells. Protein aggregates inside the cell can be intranuclear in which they are inside the nucleus or cytoplasm in which they are in the space outside of the nucleus but still within the cell membrane. The protein aggregates described in this invention are granular protein aggregates.

As used herein, the term “protein aggregate inhibiting amount” refers to an amount of compound that inhibits the formation of protein aggregates at least partially or entirely. Unless specified, the inhibition could be directed to protein aggregates inside the cell or outside the cell.

As used herein, the term “intranuclear” or “intranuclearly” refers to the space inside the nuclear compartment of an animal cell.

The term “cytoplasm” refers to the spice outside of the nucleus but within the outer cell wall of an animal cell.

As used herein, the term “pathogenic protein aggregate” refers to protein aggregates that are associated with disease conditions. These disease conditions include but are not limited to the death of a cell or the partial or complete loss of the neuronal signaling among two or more cells. Pathogenic protein aggregates can be located inside of a cell, for example, pathogenic intracellular protein aggregates or outside of a cell, for example, pathogenic extracellular protein aggregates.

The term “ocular neurotransmitter” refers to chemicals which transmit signals from a neuron to a target cell in the eye.

The term “synapse” refers to junctions between ocular neurons. These junctions allow for the passage of chemical signals from one cell to another.

The term “G protein” refers to a family of proteins involved in transmitting chemical signals outside the cell and causing changes inside of the cell. The Rho family of G proteins is small G protein, which are involved in regulating actin cytoskeletal dynamics, cell movement, motility, transcription, cell survival, and cell growth. RHOA, RAC1, and CDC42 are the most studied proteins of the Rho family. Active G proteins are localized to the cellular membrane where they exert their maximal biological effectiveness.

The terms “treat”, “treating” or “treatment”, as used herein, include alleviating, abating or ameliorating a disease or condition or one or more symptoms thereof, preventing additional symptoms, ameliorating or preventing the underlying metabolic causes of symptoms, inhibiting the disease or condition, e.g., arresting or suppressing the development of the disease or condition, relieving the disease or condition, causing regression of the disease or condition, relieving a condition caused by the disease or condition, or suppressing the symptoms of the disease or condition, and are intended to include prophylaxis. The terms also include relieving the disease or conditions, e.g., causing the regression of clinical symptoms. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the individual, notwithstanding that the individual is still be afflicted with the underlying disorder. For prophylactic benefit, the compositions are administered to an individual at risk of developing a particular disease, or to an individual reporting one or more of the physiological symptoms of a disease, even though a diagnosis of this disease has not been made.

The terms “preventing” or “prevention” refer to a reduction in risk of acquiring a disease or disorder (i.e., causing at least one of the clinical symptoms of the disease not to develop in a subject that may be exposed to or predisposed to the disease but does not yet experience or display symptoms of the disease). The terms further include causing the clinical symptoms not to develop, for example in a subject at risk of suffering from such a disease or disorder, thereby substantially averting onset of the disease or disorder.

The term “carrier” as used herein, refers to relatively nontoxic chemical compounds or agents that facilitate the incorporation of a compound into cells or tissues.

The term “axon” refers to projections of neurons that conduct signals to other cells through synapses. The term “axon growth” refers to the extension of the axon projection via the growth cone at the tip of the axon.

The term “ocular neural disease” refers to diseases that compromise the cell viability of ocular neurons.

The term “pharmaceutically acceptable”, as used herein, refers to a material, including but not limited, to a salt, carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “cyclodextrin,” as used herein, refers to cyclic carbohydrates consisting of at least six to eight sugar molecules in a ring formation. The outer part of the ring contains water soluble groups; at the center of the ring is a relatively nonpolar cavity able to accommodate small molecules.

The term “effective amount,” as used herein, refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. An appropriate “effective” amount in any individual case may be determined using techniques, such as a dose escalation study.

The term “patient”, “subject” or “individual” are used interchangeably. As used herein, they refer to individuals suffering from a disorder, and the like, encompasses mammals and non-mammals. None of the terms require that the individual be under the care and/or supervision of a medical professional. Mammals are any member of the Mammalian class, including but not limited to humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In some embodiments of the methods and compositions provided herein, the individual is a mammal. In preferred embodiments, the individual is a human.

The term “about” when used before a numerical designation, e.g., temperature, time, amount, and concentration, including range, indicates approximations which may vary by (+) or (−) 10%, 5%, or 1%.

### GGA Derivatives

GGA derivatives useful in this invention include those described in PCT publication no. WO 2012/031028 and PCT application no. PCT/US2012/027147, each of which are incorporated herein by reference in its entirety. These and other GGA derivatives provided and/or utilized herein are structurally shown below.

In one aspect, the GGA derivative provided and/or utilized herein is of Formula I:

[chemical structure]

or a tautomer or pharmaceutically acceptable salt thereof, wherein
n¹ is 1 or 2;
each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently are hydrogen or C₁-C₆ alkyl;

### Q¹ is —(C═O)—, —(C═S)—, or —S(O₂)—;

Q₂ is hydrogen, R⁶, —O—R⁶, —NR⁷R⁸, or is a chiral moiety;

### R⁶ is:

C₁-C₆ alkyl, optionally substituted with —CO₂H or an ester thereof, C₁-C₆ alkoxy, oxo, —OH, —CR═CR₂, —C≡CR, C₃-C₁₀ cycloalkyl, C₃-C₈ heterocyclyl, C₆-C₁₀aryl, C₂-C₁₀ heteroaryl, wherein each R independently is hydrogen or C₁-C₆ alkyl;

CO—C₁-C₆ alkyl;

C₃-C₁₀ cycloalkyl;

C₃-C₈ heterocyclyl;

C₆-C₁₀ aryl; or

C₂-C₁₀ heteroaryl;

wherein each cycloalkyl, heterocyclyl, aryl, or heteroaryl is optionally substituted with 1-3 alkyl groups; —CF₃, 1-3 halo, preferably, chloro or fluoro, groups; 1-3 nitro groups; 1-3 C₁-C₆ alkoxy groups; —CO-phenyl; or —NR¹⁸R¹⁹, each R¹⁸ and R¹⁹ independently is hydrogen; C₁-C₆ alkyl, optionally substituted with —CO₂H or an ester thereof, C₁-C₆ alkoxy, oxo, —CR═CR₂, —CCR, C₃-C₁₀ preferably C₃-C₈ cycloalkyl, C₃-C₈ heterocyclyl, C₆-C₁₀aryl, or C₂-C₁₀ heteroaryl, wherein each R independently is hydrogen or C₁-C₆ alkyl; C₃-C₁₀ cycloalkyl; C₃-C₈ heterocyclyl; C₆-C₁₀ aryl; or C₂-C₁₀ heteroaryl; wherein each cycloalkyl, heterocyclyl, aryl, or heteroaryl is optionally substituted with 1-3 alkyl groups, optionally substituted with 1-3 halo, preferably, fluoro, groups, where R¹⁸ and R¹⁹ together with the nitrogen atom they are attached to form a 5-7 membered heterocycle;
each R⁷ and R⁸ are independently hydrogen or defined as R⁶; and [symbol] refers to a mixture of cis and trans isomers at the corresponding position wherein at least 80% and, preferably, no more than 95% of the compound of Formula (I) is present as a trans isomer.

In one embodiment, the GGA derivative provided and/or utilized is of Formula (I-A):

[chemical structure]

as a substantially pure trans isomer around the 2,3 double bond wherein, n¹, R¹-R⁵, Q¹, and Q² are defined as in Formula (I) above.

In another embodiment, n¹ is 1. In another embodiment, n¹ is 2.

In another embodiment, the GGA derivative provided and/or utilized is of Formula (I-B):

[chemical structure]

as a substantially pure trans isomer around the 2,3 double bond wherein, R¹-R⁵, Q¹, and Q² are defined as in Formula (I) above.

In another embodiment, the GGA derivative provided and/or utilized is of Formula I-C:

[chemical structure]

wherein Q¹ and Q² are defined as in Formula (I) above.

In another embodiment, the GGA derivative provided and/or utilized is of Formula (I-D), (I-E), or (I-F):

[chemical structure]

wherein R⁶-R⁸ are defined as in Formula (I) above.

In another embodiment, the GGA derivative provided and/or utilized is of Formula (I-G), (I-H), or (I-I):

[chemical structure]

as a substantially pure trans isomer around the 2,3 double bond wherein R⁶-R⁸ are defined as in Formula (I) above.

In a preferred embodiment, R⁶ is C₆-C₁₀ aryl, such as naphthyl. In another preferred embodiment, R⁶ is a heteroaryl, such as quinolinyl.

In another aspect, the GGA derivative provided and/or utilized in this invention is of Formula (II):

[chemical structure]

or a pharmaceutically acceptable salt thereof, wherein
m is 0 or 1;
n is 0, 1, or 2;
each R¹ and R² are independently C₁-C₆ alkyl, or R¹ and R² together with the carbon atom they are attached to form a C₅-C₇ cycloalkyl ring optionally substituted with 1-3 C₃-C₆ alkyl groups;
each of R³, R⁴, and R⁵ independently are hydrogen or C₁-C₆ alkyl;
Q₃ is —OH, —NR²²R²³—X—CO—NR²⁴R²⁵, —X—CS—NR²⁴R²⁵ or —X—SO₂—NR²⁴R²⁵;

### X is —O—, —S—, —NR²⁶—, or —CR²⁷R²⁸;

each R²² and R²³ independently is hydrogen; C₁-C₆ alkyl, optionally substituted with C₁-C₆ alkoxy; and C₃-C₁₀ cycloalkyl;
each R²⁴ and R²⁵ independently is hydrogen, C₁-C₆ alkyl, optionally substituted with —CO₂H or an ester thereof, C₁-C₆ alkoxy, oxo, —OH, —CR═CR₂, —C≡CR, C₃-C₁₀ cycloalkyl, C₃-C₈ heterocyclyl, C₆-C₁₀ aryl, C₂-C₁₀ heteroaryl, wherein each R independently is hydrogen or C₁-C₆ alkyl;

C₃-C₁₀ cycloalkyl;

C₃-C₈ heterocyclyl;

C₆-C₁₀ aryl; or

C₂-C₁₀ heteroaryl;

wherein each cycloalkyl, heterocyclyl, aryl, or heteroaryl is optionally substituted with 1-3 alkyl groups; —CF₃, 1-3 halo, preferably, chloro or fluoro, groups; 1-3 nitro groups; 1-3 C₁-C₆ alkoxy groups; —CO-phenyl; or —NR¹⁸R¹⁹;
each R¹⁸ and R¹⁹ independently is hydrogen; C₁-C₆ alkyl, optionally substituted with —CO₂H or an ester thereof, C₁-C₆ alkoxy, oxo, —CR═CR₂, —CCR, C₃-C₁₀ preferably C₃-C₈ cycloalkyl, C₃-C₈ heterocyclyl, C₆-C₁₀ aryl, or C₂-C₁₀ heteroaryl, wherein each R independently is hydrogen or C₁-C₆ alkyl; C₃-C₁₀ cycloalkyl; C₃-C₈ heterocyclyl; C₆-C₁₀ aryl; or C₂-C₁₀ heteroaryl; wherein each cycloalkyl, heterocyclyl, aryl, or heteroaryl is optionally substituted with 1-3 alkyl groups, optionally substituted with 1-3 halo, preferably, fluoro, groups, where R¹⁸ and R¹⁹ together with the nitrogen atom they are attached to form a 5-7 membered heterocycle;
R²⁶ is hydrogen or together with R²⁴ or R²⁵ and the intervening atoms form a 5-7 membered heterocyclic ring optionally substituted with 1-3 C₁-C₆ alkyl groups; and each R²⁷ and R²⁸ independently are hydrogen, C₁-C₆ alkyl, —COR⁸¹ or —CO₂R⁸¹, or R²⁷ together with R²⁴ or R²⁵ and the intervening atoms form a 5-7 membered heterocyclyl ring optionally substituted with 1-3 C₁-C₆ alkyl groups.

As used herein, the compound of Formula (II) includes optical isomers such as enantiomers and diastereomers. As also used herein, an ester refers preferably to a phenyl or a C₁-C₆ alkyl ester, which phenyl or alkyl group is optionally substituted with a amino group.

In one embodiment, Q₃ is —NR²²R²³—X—CO_NR²⁴R²⁵, —X—CS—NR²⁴R²⁵, or —X—SO₂—NR²⁴R²⁵. In another embodiment, Q₃ is —X—CO—NR²⁴R²⁵, —X—CS—NR²⁴R²⁵, or —X—SO₂—NR²⁴R²⁵. In another embodiment, Q₃ is —NR²²R²³. In another embodiment, Q₃ is —OH.

In one embodiment, the compound of Formula (II) is of formula:

[chemical structure]

wherein R¹, R², R³, R⁴, R⁵, and Q₃ are defined as in any aspect or embodiment herein.

In another embodiment, the GGA derivative provided and/or utilized is of formula:

[chemical structure]

wherein R¹, R², R⁴, R⁵, and Q₃ are defined as in any aspect and embodiment here.

In one embodiment, the compound of Formula (II) is of formula:

[chemical structure]

wherein R¹, R², R³, R⁴, R⁵, and Q₃ are defined as in any aspect or embodiment herein.

In another embodiment, the GGA derivative provided and/or utilized is of formula:

[chemical structure]

wherein R¹, R², R⁴, R⁵, m, n, X, R²⁴ and R²⁵ are defined as in any aspect and embodiment here.

In another embodiment, the GGA derivative provided and/or utilized is of formula:

[chemical structure]

wherein R¹, R², R⁴, R⁵, m, n, and R²⁴ are defined as in any aspect and embodiment here.

In another embodiment, the GGA derivative provided and/or utilized is of formula:

[chemical structure]

wherein R²⁴ is defined as in any aspect and embodiment here.

In another embodiment, the GGA derivative provided and/or utilized is of formula:

[chemical structure]

wherein R²⁴ is defined as in any aspect and embodiment here.

In another embodiment, the GGA derivative provided and/or utilized is of formula:

[chemical structure]

wherein R²⁴ is defined as in any aspect and embodiment here.

In another embodiment, the GGA derivative provided and/or utilized is of formula:

[chemical structure]

wherein R²⁴ is defined as in any aspect and embodiment here.

In another embodiment, the GGA derivative provided and/or utilized is of formula:

[chemical structure]

wherein R²⁴ and R²⁵ are defined as in any aspect and embodiment here.

In another embodiment, the GGA derivative provided and/or utilized is of formula:

[chemical structure]

wherein R²⁴ is defined as in any aspect and embodiment here.

In another embodiment, the GGA derivative provided and/or utilized is of formula:

[chemical structure]

wherein R²⁴ and R²⁵ are defined as in any aspect and embodiment here.

In one embodiment, m is 0. In another embodiment, m is 1.

In another embodiment, n is 0. In another embodiment, n is 1. In another embodiment, n is 2.

In another embodiment, m+n is 1. In another embodiment, m+n is 2. In another embodiment, m+n is 3.

In another embodiment, R¹ and R² are independently C₁-C₆ alkyl. In another embodiment, R¹ and R² independently are methyl, ethyl, or isopropyl.

In another embodiment, R¹ and R² together with the carbon atom they are attached to form a C₅-C₇ cycloalkyl ring optionally substituted with 1-3 C₁-C₆ alkyl groups. In another embodiment, R¹ and R² together with the carbon atom they are attached to form a ring that is:

[chemical structure]

In another embodiment, R³, R⁴, and R⁵ are independently C₁-C₆ alkyl. In another embodiment, one of R³, R⁴, and R⁵ are alkyl, and the rest are hydrogen. In another embodiment, two of R³, R⁴, and R⁵ are alkyl, and the rest are hydrogen. In another embodiment, R³, R⁴, and R⁵ are hydrogen. In another embodiment, R³, R⁴, and R⁵ are methyl.

In another embodiment, Q₃ is —X—CO—NR²⁴R²⁵. In another embodiment, Q₃ is —X—CS—NR²⁴R²⁵. In another embodiment, Q₃ is —X—SO₂—NR²⁴R²⁵. In another embodiment, Q₃ is —OCONHR²⁴—OCONR²⁴R²⁵, NHCONHR²⁴NHCONR²⁴R²⁵, —OCSNHR²⁴, —OCSNR²⁴R²⁵, NHCSNHR²⁴, or —NHCSNR²⁴R²⁵.

In another embodiment, X is —O—. In another embodiment, X is —NR²⁶—. In another embodiment, X is or —CR²⁷R²⁸.

In another embodiment, one of R²⁴ and R²⁵ is hydrogen. In another embodiment, one or both of R²⁴ and R²⁵ are C₁-C₆ alkyl. In another embodiment, one or both of R²⁴ and R²⁵ are C₁-C₆ alkyl, optionally substituted with an R²⁰ group, wherein R²⁰ is —CO₂H or an ester thereof, C₁-C₆ alkyl, C₃-C₁₀ cycloalkyl, C₃-C₈ heterocyclyl, C₆-C₁₀ aryl, or C₂-C₁₀ heteroaryl. In another embodiment, one or both of R²⁴ and R²⁵ are C₃-C₁₀ cycloalkyl. In another embodiment, one or both of R²⁴ and R²⁵ are C₃-C₁₀ cycloalkyl substituted with 1-3 alkyl groups. In another embodiment, one or both of R²⁴ and R²⁵ are C₃-C₈ heterocyclyl. In another embodiment, one or both of R²⁴ and R²⁵ are C₆-C₁₀ aryl. In another embodiment, one or both of R²⁴ and R²⁵ are C₂-C₁₀ heteroaryl. In another embodiment, R²⁴ and R²⁵ together with the nitrogen atom they are attached to form a 5-7 membered heterocycle.

In another embodiment, R²⁰ is —CO₂H or an ester thereof. In another embodiment, R²⁰ is C₁-C₆ alkyl. In another embodiment, R²⁰ is C₃-C₁₀ cycloalkyl. In another embodiment, R²⁰ is C₃-C₈ heterocyclyl. In another embodiment, R²⁰ is C₆-C₁₀ aryl. In another embodiment, R²⁰ is or C₂-C₁₀ heteroaryl.

In another embodiment, the GGA derivative provided and/or utilized is of formula (II):

[chemical structure]

or a pharmaceutically acceptable salt thereof, wherein
   m is 0 or 1;
   n is 0, 1, or 2;
each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently are hydrogen or C₁-C₆ alkyl;
Q₃ is —X—CO—NR²⁴R²⁵ or —X—SO₂—NR²⁴R²⁵;
X is —O—, —NR²⁶—, or —CR²⁷R²⁸;
R²⁶ is hydrogen or together with R²⁴ or R²⁵ and the intervening atoms form a 5-7 membered ring optionally substituted with 1-3 C₁-C₆ alkyl groups;
each R²⁷ and R²⁸ independently are hydrogen, C₁-C₆ alkyl, —COR⁸¹ or —CO₂R⁸¹, or R²⁷ together with R²⁴ or R²⁵ and the intervening atoms form a 5-7 membered cycloalkyl or heterocyclyl ring optionally substituted with 1-3 C₁-C₆ alkyl groups;
each R²⁴ and R²⁵ independently is
hydrogen,
C₁-C₆ alkyl, optionally substituted with —CO₂H or an ester thereof, C₃-C₁₀ preferably C₃-C₈ cycloalkyl, C₃-C₈ heterocyclyl, C₆-C₁₀ aryl, or C₂-C₁₀ heteroaryl,
C₃-C₁₀ cycloalkyl,
C₃-C₈ heterocyclyl,
C₆-C₁₀ aryl, or
C₂-C₁₀ heteroaryl,
   wherein each cycloalkyl, heterocyclyl, aryl, or heteroaryl is optionally substituted with 1-3 C₁-C₆ alkyl groups, or R²⁴ and R²⁵ together with the nitrogen atom they are attached to form a 5-7 membered heterocycle.

In another embodiment, provided herein are compounds of formula:

[chemical structure]

In another aspect, the GGA derivative provided and/or utilized herein is of Formula III:

[chemical structure]

or a pharmaceutically acceptable salt of each thereof, wherein

m is 0 or 1;

n is 0, 1, or 2;

each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;

each of R³, R⁴, and R⁵ independently are hydrogen or C₁-C₆ alkyl;

Q₄ is selected from the group consisting of:

[chemical structure]

when X¹ is bonded via a single bond, X¹ is —O—, —NR³¹—, or —CR³²R³³—, and when X¹ is bonded via a double bond, X¹ is —CR³²—;

Y¹ is hydrogen, —OH or —O—R¹⁰, Y² is —OH, —OR¹¹ or —NHR¹², or Y¹ and Y² are joined to form an oxo group (═O), an imine group (═NR¹³), a oxime group (═N—OR¹⁴), or a substituted or unsubstituted vinylidene (═CR¹⁶R¹⁷);

R³⁰ is C₁-C₆ alkyl optionally substituted with 1-3 alkoxy or 1-5 halo group, Cᵣ C₆ alkenyl, C₂-C₆ alkynyl, C₃-C₁₀ cycloalkyl, C₆-C₁₀ aryl, C₃-C₈heterocyclyl, or C₂-C₁₀ heteroaryl, wherein each cycloalkyl or heterocyclyl is optionally substituted with 1-3 C₁-C₆ alkyl groups, or wherein each aryl or heteroaryl is independently substituted with 1-3 C₁-C₆ alkyl or nitro groups, or R³⁰ is —NR³⁴R³⁵;

R³¹ is hydrogen or together with R³⁰ and the intervening atoms form a 5-7 membered ring optionally substituted with 1-3 C₁-C₆ alkyl groups;

each R³² and R³³ independently are hydrogen, C₁-C₆ alkyl, —COR⁸¹ or —CO₂R⁸¹, or R³² together with R³⁰ and the intervening atoms form a 5-7 membered cycloalkyl or heterocyclyl ring optionally substituted with oxo or 1-3 C₁-C₆ alkyl groups;

R¹⁰ is C₁-C₆ alkyl;

R¹¹ and R¹² are independently C₁-C₆ alkyl, C₃-C₁₀ cycloalkyl, —CO₂R¹⁵, or —CON(R¹⁵)₂, or R¹⁰ and R¹¹ together with the intervening carbon atom and oxygen atoms form a heterocycle optionally substituted with 1-3 C₁-C₆ alkyl groups;

R¹³ is C₁-C₆ alkyl or C₃-C₁₀ cycloalkyl optionally substituted with 1-3 C₁-C₆ alkyl groups;

R¹⁴ is hydrogen, C₃-C₈ heterocyclyl, or C₁-C₆ alkyl optionally substituted with a —CO₂H or an ester thereof or a C₆-C₁₀ aryl, C₂-C₆ alkenyl, C₂-C₆ alkynyl, C₃-C₁₀ cycloalkyl, or a C₃-C₈ heterocyclyl, wherein each cycloalkyl, heterocyclyl, or aryl, is optionally substituted with 1-3 alkyl groups;

each R¹⁵ independently are hydrogen, C₃-C₁₀ cycloalkyl, C₁-C₆ alkyl optionally substituted with 1-3 substituents selected from the group consisting of —CO₂H or an ester thereof, aryl, or C₃-C₈ heterocyclyl, or two R¹⁵ groups together with the nitrogen atom they are bonded to form a 5-7 membered heterocycle;

R¹⁶ is hydrogen or C₁-C₆ alkyl;

R¹⁷ is hydrogen, C₁-C₆ alkyl substituted with 1-3 hydroxy groups, —CHO, or is CO₂H or an ester thereof;

each R³⁴ and R³⁵ independently is hydrogen, C₁-C₆ alkyl, optionally substituted with —CO₂H or an ester thereof, C₃-C₁₀ cycloalkyl, C₃-C₈ heterocyclyl, C₆-C₁₀ aryl, or C₂-C₁₀ heteroaryl, or is C₃-C₁₀ cycloalkyl, C₃-C₈ heterocyclyl, C₆-C₁₀ aryl, or C₂-C₁₀ heteroaryl, wherein each cycloalkyl, heterocyclyl, aryl, or heteroaryl is optionally substituted with 1-3 alkyl groups, or R³⁴ and R³⁵ together with the nitrogen atom they are attached to form a 5-7 membered heterocycle; and

each R⁸¹ independently is C₁-C₆ alkyl.

In one embodiment, m is 0. In another embodiment, m is 1. In another embodiment, n is 0. In another embodiment, n is 1. In another embodiment, n is 2.

In one embodiment, the compound of Formula (III) is of formula:

[chemical structure]

wherein Q₄, R¹, R², R³, R⁴, R⁵, R³⁰, X¹, Y¹, and Y² are defined as in any aspect or embodiment herein.

In one embodiment, the GGA derivative provided and/or utilized is of formula:

[chemical structure]

wherein R¹, R², R³, R⁴, R⁵, R³⁰, X¹, Y¹, and Y² are defined as in any aspect and embodiment here.

In another embodiment, the GGA derivative provided and/or utilized is of formula:

[chemical structure]

wherein R¹, R², R³, R⁴, R⁵, R³⁰, X¹, and Y² are defined as in any aspect and embodiment herein.

In another embodiment, the GGA derivative provided and/or utilized is of formula:

[chemical structure]

wherein R¹, R², R³, R⁴, R⁵, R³⁰ and X¹ are defined as in any aspect and embodiment herein.

In another embodiment, the GGA derivative provided and/or utilized is of formula:

[chemical structure]

wherein R¹, R², R⁴, R⁵, and Q₄ are defined as in any aspect and embodiment herein.

In another embodiment, the GGA derivative provided and/or utilized is of formula:

[chemical structure]

wherein R¹, R², R⁴, R⁵, m, n, X¹, and R³⁰ are defined as in any aspect and embodiment here.

In another embodiment, the GGA derivative provided and/or utilized is of formula:

[chemical structure]

wherein R¹, R², R⁴, R⁵, m, n, and R³⁴ are defined as in any aspect and embodiment here.

In another embodiment, the GGA derivative provided and/or utilized is of formula:

[chemical structure]

wherein R¹, R², R⁴, R⁵, R³⁰, m, n, and R¹⁵ are defined as in any aspect and embodiment here.

In another embodiment, each R¹ and R² are C₁-C₆ alkyl. In another embodiment, each R¹ and R² are methyl, ethyl, or isopropyl. In another embodiment, R¹ and R² together with the carbon atom they are attached to form a 5-6 membered ring optionally substituted with 1-3 C₁-C₆ alkyl groups. In another embodiment, R¹ and R² together with the carbon atom they are attached to form a ring that is:

[chemical structure]

In another embodiment, R³, R⁴, and R⁵ are C₁-C₆ alkyl. In another embodiment, one of R³, R⁴, and R⁵ are alkyl, and the rest are hydrogen. In another embodiment, two of R³, R⁴, and R⁵ are alkyl, and the rest are hydrogen. In another embodiment, R³, R⁴, and R⁵ are hydrogen. In another embodiment, R³, R⁴, and R⁵ are methyl.

In another embodiment, X¹ is O. In another embodiment, X¹ is —NR³¹. In another embodiment, R³¹ is hydrogen. In another embodiment, R³¹ together with R³⁰ and the intervening atoms form a 5-7 membered ring optionally substituted with 1-3 C₁-C₆ alkyl groups. In another embodiment, X¹ is —CR³²R³³—. In another embodiment, X¹ is —CR³²—. In another embodiment, each R³² and R³³ independently are hydrogen, C₁-C₆ alkyl, —COR⁸¹, or —CO₂R⁸¹. In another embodiment, R³² is hydrogen, and R³³ is hydrogen, C₁-C₆ alkyl, —COR⁸¹, or —CO₂R⁸¹.

In another embodiment, R³³ is hydrogen. In another embodiment, R³³C₁-C₆ alkyl. In another embodiment, R³³ is methyl. In another embodiment, R³³ is —CO₂R⁸¹. In another embodiment, R³³ is —COR⁸¹.

In another embodiment, R³² together with R³⁰ and the intervening atoms form a 5-7 membered ring. In another embodiment, the moiety:

[chemical structure]

which is “Q₄,” has the structure:

[chemical structure]

wherein R³³ is hydrogen, C₁-C₆ alkyl, or —CO₂R⁸¹ and n is 1, 2, or 3. Within these embodiments, in certain embodiments, R³³ is hydrogen or C₁-C₆ alkyl. In one embodiment, R³³ is hydrogen. In another embodiment, R³³ is C₁-C₆ alkyl.

In another embodiment, R³⁰ is C₁-C₆ alkyl. In another embodiment, R³⁰ is methyl, ethyl, butyl, isopropyl, or tertiary butyl. In another embodiment, R³⁰ is C₁-C₆ alkyl substituted with 1-3 alkoxy or 1-5 halo group. In another embodiment, R³⁰ is alkyl substituted with an alkoxy group. In another embodiment, R³⁰ is alkyl substituted with 1-5, preferably, 1-3, halo, preferably fluoro, groups.

In another embodiment, R³⁰ is NR³⁴R³⁵. In a preferred embodiment, R³⁵ is H. In a preferred embodiment, R³⁴ is C₁-C₆ alkyl, optionally substituted with a group selected from the group consisting of —CO₂H or an ester thereof, C₃-C₁₀ cycloalkyl, C₃-C₈ heterocyclyl, C₆-C₁₀ aryl, or C₂-C₁₀ heteroaryl. In another preferred embodiment, R³⁴ is C₃-C₁₀ cycloalkyl, C₃-C₈ heterocyclyl, C₆-C₁₀ aryl, or C₂-C₁₀ heteroaryl. In a more preferred embodiment, R³⁴ is C₃-C₁₀ cycloalkyl.

In another embodiment, R³⁰ is C₂-C₆ alkenyl or C₂-C₆ alkynyl. In another embodiment, R³⁰ is C₃-C₁₀ cycloalkyl. In another embodiment, R³⁰ is C₃-C₁₀ cycloalkyl substituted with 1-3 C₁-C₆ alkyl groups. In another embodiment, R³⁰ is cyclopropyl, cyclobutyl, cyclopentyl, cyclohexyl, or adamentyl. In another embodiment, R³⁰ is C₆-C₁₀ aryl or C₂-C₁₀ heteroaryl. In another embodiment, R³⁰ is a 5-7 membered heteroaryl containing at least 1 oxygen atom. In another embodiment, R³⁰ is C₆-C₁₀ aryl, C₃-C₈ heterocyclyl, or C₂-C₁₀ heteroaryl, wherein each aryl, heterocyclyl, or heteroaryl is optionally substituted with 1-3 C₁-C₆ alkyl groups.

In another embodiment, Y² is —O—R¹¹. In another embodiment, Y¹ and Y² are joined to form ═NR¹³. In another embodiment, Y¹ and Y² are joined to form ═NOR¹⁴. In another embodiment, Y¹ and Y² are joined to form (═O). In another embodiment, Y¹ and Y² are joined to form ═CR¹⁶R¹⁷.

In another embodiment, Q₄ is —CR³³COR³⁰. In another embodiment, R³⁰ is C₁-C₆ alkyl optionally substituted with an alkoxy group. In another embodiment, R³⁰ is C₃-C₈ cycloalkyl. In another embodiment, R³³ is hydrogen. In another embodiment, R³³ is C₁-C₆ alkyl. In another embodiment, R³³ is CO₂R⁸¹. In another embodiment, R³³ is COR⁸¹.

In another embodiment, Q₄ is —CH₂—CH(O—CONHR¹⁵)—R³⁰. In another embodiment, R¹⁵ is C₃-C₈ cycloalkyl. In another embodiment, R¹⁵ is C₁-C₆ alkyl optionally substituted with 1-3 substituents selected from the group consisting of —CO₂H or an ester thereof, aryl, or C₃-C₈ heterocyclyl. In a preferred embodiment within these embodiments, R³⁰ is C₁-C₆ alkyl.

In another embodiment, Q₄ is —O—CO—NHR³⁴ within these embodiment, in another embodiment, R³⁴ is C₁-C₆ alkyl, optionally substituted with —CO₂H or an ester thereof, C₃-C₈ cycloalkyl, C₃-C₈ heterocyclyl, C₂-C₁₀ aryl, or C₂-C₁₀ heteroaryl. In yet another embodiment, R³⁴ is C₃-C₈ cycloalkyl, C₃-C₈ heterocyclyl, C₂-C₁₀ aryl, or C₂-C₁₀ heteroaryl.

In another embodiment, R¹⁴ is hydrogen. In another embodiment, R¹⁴ is C₁-C₆ alkyl optionally substituted with a —CO₂H or an ester thereof or a C₆-C₁₀ aryl optionally substituted with 1-3 alkyl groups. In another embodiment, R¹⁴ is C₂-C₆ alkenyl. In another embodiment, R¹⁴ is C₂-C₆ alkynyl In another embodiment, R¹⁴ is C₃-C₆ cycloalkyl optionally substituted with 1-3 alkyl groups. In another embodiment, R¹⁴ is C₃-C₈ heterocyclyl optionally substituted with 1-3 alkyl groups.

In another embodiment, preferably, R¹⁶ is hydrogen. In another embodiment, R¹⁷ is CO₂H or an ester thereof. In another embodiment, R¹⁷ is C₁-C₆ alkyl substituted with 1-3 hydroxy groups. In another embodiment, R¹⁷ is C₁-C₃ alkyl substituted with 1 hydroxy group. In another embodiment, R¹⁷ is —CH₂OH.

In another embodiment, R¹⁰ and R¹¹ together with the intervening carbon atom and oxygen atoms form a heterocyle of formula:

[chemical structure]

wherein q is 0 or 1, p is 0, 1, 2, or 3, and R³⁶ is C₁-C₆ alkyl.

In another embodiment, q is 1. In another embodiment, q is 2. In another embodiment, p is 0. In another embodiment, p is 1. In another embodiment, p is 2. In another embodiment, p is 3.

In one aspect, the GGA derivative provided and/or utilized herein is of Formula (IV):

[chemical structure]

or a tautomer thereof, or a pharmaceutically acceptable salt of each thereof, wherein
m is 0 or 1;
n is 0, 1, or 2;
each R¹ and R² are independently C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently are hydrogen or C₁-C₆ alkyl, or R⁵ and Q₅ together with the intervening carbon atoms form a 6 membered aryl ring, or a 5-8 membered cycloalkenyl ring, or a 5-14 membered heteroaryl or heterocycle, wherein each aryl, cycloalkenyl, heteroaryl, or heterocycle, ring is optionally substituted with 1-2 substituents selected from the group consisting of halo, hydroxy, oxo, —N(R⁴⁰)₂, and C₁-C₆ alkyl group;
Q₅ is —C(═O)H, —CO₂H or —CH═CHCO₂H, or a C₁-C₆ alkyl ester or acyl halide thereof, wherein the ester is optionally substituted with —CO-phenyl; a 6-10 membered aryl or a 5-14 membered heteroaryl or heterocycle containing up to 6 ring heteroatoms, wherein the heteroatom is selected from the group consisting of O, N, S, and oxidized forms of N and S, and further wherein the aryl, heteroaryl, or heterocyclyl ring is optionally substituted with 1-3 substituents selected from the group consisting of:

hydroxy, oxo, —N(R⁴⁰)₂, C₁-C₆ alkoxy group, and C₁-C₆ alkyl group, wherein the alkyl group is optionally substituted with 1-3 substituents selected from hydroxy, NH₂, C₆-C₁₀ aryl, —CO₂H or an ester or an amide thereof,

a 5-9 membered heteroaryl containing up to 3 ring heteroatoms, wherein the heteroaryl is optionally substituted with 1-3 hydroxy, —N(R⁴⁰)₂, and C₁-C₆ alkyl group,

benzyl, and phenyl optionally substituted with 1-3 substituents selected from the group consisting of C₁-C₆ alkyl, C₁-C₆ alkoxy, hydroxy, and halo groups; and wherein each R⁴⁰ independently is hydrogen or C₁-C₆ alkyl.

As used herein, the compound of Formula (IV) includes tautomers and optical isomers such as enantiomers and diastereomers. As also used herein, an ester refers preferably to a phenyl or a C₁-C₆ alkyl ester, which phenyl or alkyl group is optionally substituted with a amino group. As used herein, an amide refers preferably to a moiety of formula —CON(R⁴⁰)₂, wherein R⁴⁰ is defined as above.

In some embodiment, Q₆ is selected from a group consisting of oxazole, oxadiazole, oxazoline, azalactone, imidazole, diazole, triazole, and thiazole, wherein each heteroaryl or heterocycle is optionally substituted as disclosed above.

In one embodiment, the GGA derivative provided and/or utilized is of formula IV-A:

[chemical structure]

In another embodiment, the GGA derivative provided and/or utilized is of formula IV-B:

[chemical structure]

wherein R¹, R², R⁴, R⁵, and Q₅ are defined as in any aspect and embodiment here.

In another embodiment, Q₅ is selected from the group consisting of:

[chemical structure]

wherein R¹¹ is C₁-C₆ alkyl, C₆-C₁₀ aryl, C₃-C₈ heteroaryl, C₃-C₈ heteroaryl, C₃-C₁₀ cycloalkyl, and the alkyl group is optionally substituted with 1-3 C₆-C₁₀ aryl, C₃-C₈ heteroaryl, C₃-C₈ heteroaryl, C₃-C₁₀ cycloalkyl groups, and the aryl, heteroaryl, heteroaryl, cycloalkyl groups are optionally substituted with 1-3 C₁-C₆ alkyl, C₁-C₆ alkoxy, halo, preferably chloro or fluoro, C₆-C₁₀ aryl, C₃-C₈ heteroaryl, C₃-C₈ heteroaryl, C₃-C₁₀ cycloalkyl group. In another embodiment, Q₅ is phenyl, optionally substituted as described herein. In another embodiment, Q₅ is benzimidazole, benzindazole, and such other 5-6 fused 9-membered bicyclic heteroaryl or heterocycle. In another embodiment, Q₅ is quinoline, isoquinoline, and such other 6-6 fused 10 membered heteroaryl or heterocycle. In another embodiment, Q₅ is benzodiazepine or a derivative thereof, such as, a benzodiazepinone. Various benzodiazepine and derivatives thereof are well known to the skilled artisan.

In another embodiment, m is 0. In another embodiment, m is 1.

In another embodiment, n is 0. In another embodiment, n is 1. In another embodiment, n is 2.

In another embodiment, m+n is 1: In another embodiment, m+n is 2. In another embodiment, m+n is 3.

In another embodiment, R¹ and R² are independently C₁-C₆ alkyl. In another embodiment, R¹ and R² independently are methyl, ethyl, or isopropyl.

In another embodiment, R¹ and R² together with the carbon atom they are attached to form a C₅-C₇ cycloalkyl ring optionally substituted with 1-3 C₁-C₆ alkyl groups. In another embodiment, R¹ and R² together with the carbon atom they are attached to form a ring that is:

[chemical structure]

In another embodiment, R³, R⁴, and R⁵ are independently C₁-C₆ alkyl. In another embodiment, one of R³, R⁴, and R⁵ are alkyl, and the rest are hydrogen. In another embodiment, two of R³, R⁴, and R⁵ are alkyl, and the rest are hydrogen. In another embodiment, R³, R⁴, and R⁵ are hydrogen. In another embodiment, R³, R⁴, and R⁵ are methyl.

In another embodiment, this invention provides a compound selected from the group consisting of:

[chemical structure]

wherein R¹¹ is defined as above.

In another aspect, GGA derivatives provided and/or utilized herein are of formula (V):

[chemical structure]

or a pharmaceutically acceptable salt thereof, wherein
m is 0 or 1;
n is 0, 1, or 2;
each R¹ and R² independently are C₁-C₆ alkyl, or R¹ and R² together with the carbon atom they are attached to form a C₅-C₇ cycloalkyl ring optionally substituted with 1-3 C₁-C₆ alkyl groups;
each of R³, R⁴, and R⁵ independently is hydrogen or C₁-C₆ alkyl;
Q₆ is selected from the group consisting of:

[chemical structure]

when X² is bonded via a single bond, X² is —O—, —NR⁵²—, or —CR⁵³R⁵⁴—, and when X² is bonded via a double bond, X² is —CR⁵³—;
Y¹¹ is hydrogen, —OH or —OR⁵⁵;
Y²² is OH, —OR⁵⁶, —NHR⁵⁷, or —O—CO—NR⁵⁸R⁵⁹, or Y¹¹ and Y²² are joined to form an oxo group (═O), an imine group (═NR⁶⁰), a oxime group (═N—OR⁶¹), or a substituted or unsubstituted vinylidene (═CR⁶³R⁶⁴);
R⁵¹ is C₁-C₆ alkyl, C₁-C₆ alkyl substituted with 1-3 alkoxy or 1-5 halo groups, C₂-C₆ alkenyl, C₂-C₆ alkynyl, C₃-C₁₀ cycloalkyl, C₃-C₈ heterocyclyl, C₆-C₁₀ aryl, C₂-C₁₀ heteroaryl, or —NR⁶⁵R⁶⁶, wherein each cycloalkyl or heterocyclyl is optionally substituted with 1-3 C₁-C₆ alkyl groups, and wherein each aryl or heteroaryl is optionally substituted independently with 1-3 nitro and C₁-C₆ alkyl groups;
R⁵² is hydrogen or together with R⁵¹ and the intervening atoms form a 5-7 membered ring optionally substituted with 1-3 C₁-C₆ alkyl groups;
each R⁵³ and R⁵⁴ independently are hydrogen, C₁-C₆ alkyl, —COR⁸¹, —CO₂R⁸¹, or —CONHR⁸², or R⁵³ together with R⁵¹ and the intervening atoms form a 5-7 membered cycloalkyl or heterocyclyl ring optionally substituted with 1-3 C₁-C₆ alkyl groups;
R⁵⁵ is C₁-C₆ alkyl;
each R⁵⁶ and R⁵⁷ independently are C₁-C₆ alkyl, C₃-C₁₀ cycloalkyl, —CO₂R⁶², or —CON(R⁶²)₂; or R⁵⁵ and R⁵⁶ together with the intervening carbon atom and oxygen atoms form a heterocycle optionally substituted with 1-3 C₁-C₆ alkyl groups;
R⁵⁸ is: C₃-C₁₀ cycloalkyl, C₁-C₆ alkyl optionally substituted with —OH, CO₂H or an ester thereof, or C₃-C₁₀ cycloalkyl,

[chemical structure]

R⁵⁹ is hydrogen or C₁-C₆ alkyl;
R⁶⁰ is C₁-C₆ alkyl or C₃-C₁₀ cycloalkyl optionally substituted with 1-3 C₁-C₆ alkyl groups, or is:

[chemical structure]

R⁶¹ is hydrogen, C₃-C₈ heterocyclyl, or C₁-C₆ alkyl optionally substituted with a —CO₂H or an ester thereof or a C₆-C₁₀ aryl, C₂-C₆ alkenyl, C₂-C₆ alkynyl, C₃-C₁₀ cycloalkyl, or a C₃-C₈ heterocyclyl, wherein each cycloalkyl, heterocyclyl, or aryl, is optionally substituted with 1-3 alkyl groups;
each R⁶² independently are hydrogen, C₃-C₁₀ cycloalkyl, C₁-C₆ alkyl optionally substituted with 1-3 substituents selected from the group consisting of —CO₂H or an ester thereof, aryl, C₃-C₈ heterocyclyl, or two R⁶² groups together with the nitrogen atom they are bonded to form a 5-7 membered heterocycle;
R⁶³ is hydrogen or C₁-C₆ alkyl;
R⁶⁴ is hydrogen, C₁-C₆ alkyl substituted with 1-3 hydroxy groups, —CHO, or is CO₂H or an ester thereof;
one or both of R⁶⁵ and R⁶⁶ independently are hydrogen, C₁-C₆ alkyl, optionally substituted with —CO₂H or an ester thereof, C₃-C₁₀ cycloalkyl, C₃-C₈ heterocyclyl, C₂-C₁₀ aryl, or C₂-C₁₀ heteroaryl, or is C₃-C₁₀ cycloalkyl, C₃-C₈ heterocyclyl, C₆-C₁₀aryl, or C₂-C₁₀ heteroaryl, wherein each cycloalkyl, heterocyclyl, aryl, or heteroaryl is optionally substituted with 1-3 alkyl groups, or R⁶⁵ and R⁶⁶ together with the nitrogen atom they are bonded to form a 5-7 membered heterocycle, and if only one of R⁶⁵ and R⁶⁶ are defined as above, then the other one is

[chemical structure]

and
R⁸¹ is C₁-C₆ alkyl; and
R⁸² is:

[chemical structure]

provided that, when X² is bonded via a single bond, and R⁵³ or R⁵⁴ is not —CONHR⁸², Y¹¹ and Y²² are joined to form an imine group (═NR⁶⁰), and R⁶⁰ is:

[chemical structure]

or Y²² is —O—CO—NR⁵⁸R⁵⁹;
or provided that, when Q₆ i

[chemical structure]

and R⁵³ is not —CONHR⁸², Y²² is —O—CO—NR⁵⁸R⁵⁹;
   or provided that, when Q₆ is —O—CO—NR⁶⁵R⁶⁶, then at least one of R⁶⁵ and R⁶⁶ is:

[chemical structure]

In one embodiment, the GGA derivative provided and/or utilized are of formula:

[chemical structure]

In another aspect, the GGA derivatives useful according to this invention is selected from:

[chemical structure]

In one embodiment, the compounds provided herein excludes the compound of formula:

[chemical structure]

wherein L is 0, 1, 2, or 3, and R¹⁷ is CO₂H or an ester thereof, or is —CH₂OH, or is a C₁-C₆ alkyl ester of —CH₂OH.

In another embodiment, examples of compounds provided and/or utilized by this invention include certain compounds tabulated below. Compound ID numbers in Table 1 refer to synthetic schemes in Example 7.

TABLE 1

| Compound | |
|---|---|
| ID | Structure |
| 1 | [chemical structure] |
| 2a | [chemical structure] |
| 2b | [chemical structure] |
| 2c | [chemical structure] |
| 2d | [chemical structure] |
| 2e | [chemical structure] |
| 2f | [chemical structure] |
| 2g | [chemical structure] |
| 2h | [chemical structure] |
| 2i | [chemical structure] |
| 2j | [chemical structure] |
| 2k | [chemical structure] |
| 2l | [chemical structure] |
| 4a | [chemical structure] |
| 4b | [chemical structure] |
| 4c | [chemical structure] |
| 6a | [chemical structure] |
| 6b | [chemical structure] |
| 7a | [chemical structure] |
| 7b | [chemical structure] |
| 7c | [chemical structure] |
| 7d | [chemical structure] |
| 7e | [chemical structure] |
| 7f | [chemical structure] |
| 7g | [chemical structure] |
| 7h | [chemical structure] |
| 7i | [chemical structure] |
| 7j | [chemical structure] |
| 7k | [chemical structure] |
| 7l | [chemical structure] |
| 7m | [chemical structure] |
| 7n | [chemical structure] |
| 7o | [chemical structure] |
| 7p | [chemical structure] |
| 7q | [chemical structure] |
| 7r | [chemical structure] |
| 7s | [chemical structure] |
| 7t | [chemical structure] |
| 7u | [chemical structure] |
| 7v | [chemical structure] |
| 7w | [chemical structure] |
| 7x | [chemical structure] |
| 7y | [chemical structure] |
| 7z | [chemical structure] |
| 7aa | [chemical structure] |
| 8a | [chemical structure] |
| 8b | [chemical structure] |
| 8c | [chemical structure] |
| 8d | [chemical structure] |
| 8e | [chemical structure] |
| 8f | [chemical structure] |
| 8g | [chemical structure] |
| 8h | [chemical structure] |
| 8i | [chemical structure] |
| 8j | [chemical structure] |
| 8k | [chemical structure] |
| 8l | [chemical structure] |
| 8m | [chemical structure] |
| 8n | [chemical structure] |
| 8o | [chemical structure] |
| 9a | [chemical structure] |
| 9b | [chemical structure] |
| 9c | [chemical structure] |
| 9d | [chemical structure] |
| 9e | [chemical structure] |
| 9f | [chemical structure] |
| 9g | [chemical structure] |
| 9h | [chemical structure] |
| 9i | [chemical structure] |
| 9j | [chemical structure] |
| 9k | [chemical structure] |
| 10a | [chemical structure] |
| 10b | [chemical structure] |
| 10c | [chemical structure] |
| 10d | [chemical structure] |
| 10e | [chemical structure] |
| 10f | [chemical structure] |
| 10g | [chemical structure] |
| 10h | [chemical structure] |
| 10i | [chemical structure] |
| 10j | [chemical structure] |
| 10k | [chemical structure] |
| 10l | [chemical structure] |
| 10m | [chemical structure] |
| 12 | [chemical structure] |
| 14 | [chemical structure] |
| 15 | [chemical structure] |
| 16 | [chemical structure] |
| 17a | [chemical structure] |
| 17b | [chemical structure] |
| 17c | [chemical structure] |
| 17d | [chemical structure] |
| 17e | [chemical structure] |
| 19 | [chemical structure] |
| 20a | [chemical structure] |
| 20b | [chemical structure] |
| 20c | [chemical structure] |
| 20d | [chemical structure] |
| 20e | [chemical structure] |
| 20f | [chemical structure] |
| 20g | [chemical structure] |
| 20h | [chemical structure] |
| 20i | [chemical structure] |
| 20j | [chemical structure] |
| 22 | [chemical structure] |
| 23a | [chemical structure] |
| 23b | [chemical structure] |
| 23c | [chemical structure] |
| 23d | [chemical structure] |
| 23e | [chemical structure] |
| 23f | [chemical structure] |
| 23g | [chemical structure] |
| 24 | [chemical structure] |
| 25 | [chemical structure] |
| 27a | [chemical structure] |
| 27b | [chemical structure] |
| 27c | [chemical structure] |
| 27d | [chemical structure] |
| 27e | [chemical structure] |
| 27f | [chemical structure] |
| 27g | [chemical structure] |
| 29a | [chemical structure] |
| 29b | [chemical structure] |
| 29c | [chemical structure] |
| 29d | [chemical structure] |
| 29e | [chemical structure] |
| 29f | [chemical structure] |
| 31 | [chemical structure] |
| 32 | [chemical structure] |
| 35a | [chemical structure] |
| 35b | [chemical structure] |
| 35c | [chemical structure] |
| 35d | [chemical structure] |
| 37a | [chemical structure] |
| 37b | [chemical structure] |
| 37c | [chemical structure] |
| 37d | [chemical structure] |
| 38a | [chemical structure] |
| 38b | [chemical structure] |
| 39 | [chemical structure] |
| 40a | [chemical structure] |
| 40b | [chemical structure] |
| 41 | [chemical structure] |
| 42 | [chemical structure] |
| 43 | [chemical structure] |

In another embodiment, examples of compounds provided and/or utilized by this invention include certain compounds tabulated below.

TABLE 2

| Compound | |
|---|---|
| ID | Chemical Structure |
| 51 | [chemical structure] |
| 52 | [chemical structure] |
| 54 | [chemical structure] |
| 55 | [chemical structure] |
| 56 | [chemical structure] |
| 57 | [chemical structure] |
| 58 | [chemical structure] |
| 59 | [chemical structure] |
| 60 | [chemical structure] |
| 61 | [chemical structure] |
| 62 | [chemical structure] |
| 63 | [chemical structure] |
| 64 | [chemical structure] |
| 65 | [chemical structure] |
| 66 | [chemical structure] |
| 67 | [chemical structure] |
| 68 | [chemical structure] |
| 69 | [chemical structure] |
| 70 | [chemical structure] |
| 71 | [chemical structure] |
| 72 | [chemical structure] |
| 73 | [chemical structure] |
| 74 | [chemical structure] |
| 75 | [chemical structure] |
| 76 | [chemical structure] |
| 77 | [chemical structure] |
| 78 | [chemical structure] |
| 6979 | [chemical structure] |
| 80 | [chemical structure] |
| 81 | [chemical structure] |
| 82 | [chemical structure] |
| 83 | [chemical structure] |
| 84 | [chemical structure] |
| 85 | [chemical structure] |
| 86 | [chemical structure] |
| 87 | [chemical structure] |
| 88 | [chemical structure] |
| 89 | [chemical structure] |
| 90 | [chemical structure] |
| 91 | [chemical structure] |
| 92 | [chemical structure] |
| 93 | [chemical structure] |
| 94 | [chemical structure] |
| 95 | [chemical structure] |
| 96 | [chemical structure] |
| 97 | [chemical structure] |
| 98 | [chemical structure] |
| 99 | [chemical structure] |
| 100 | [chemical structure] |
| 101 | [chemical structure] |
| 102 | [chemical structure] |
| 103 | [chemical structure] |
| 104 | [chemical structure] |
| 105 | [chemical structure] |
| 106 | [chemical structure] |
| 107 | [chemical structure] |
| 108 | [chemical structure] |
| 109 | [chemical structure] |
| 110 | [chemical structure] |
| 111 | [chemical structure] |
| 112 | [chemical structure] |
| 113 | [chemical structure] |
| 114 | [chemical structure] |
| 115 | [chemical structure] |
| 116 | [chemical structure] |
| 117 | [chemical structure] |
| 118 | [chemical structure] |
| 119 | [chemical structure] |
| 120 | [chemical structure] |
| 121 | [chemical structure] |
| 122 | [chemical structure] |
| 123 | [chemical structure] |
| 124 | [chemical structure] |
| 125 | [chemical structure] |
| 126 | [chemical structure] |
| 127 | [chemical structure] |
| 128 | [chemical structure] |
| 129 | [chemical structure] |
| 130 | [chemical structure] |
| 131 | [chemical structure] |
| 132 | [chemical structure] |
| 133 | [chemical structure] |
| 134 | [chemical structure] |
| 135 | [chemical structure] |
| 136 | [chemical structure] |
| 137 | [chemical structure] |
| 138 | [chemical structure] |
| 139 | [chemical structure] |
| 140 | [chemical structure] |
| 141 | [chemical structure] |
| 142 | [chemical structure] |
| 143 | [chemical structure] |
| 144 | [chemical structure] |
| 145 | [chemical structure] |
| 146 | [chemical structure] |
| 147 | [chemical structure] |

Illustrative and nonlimiting anticancer agents and conjugates and their methods of synthesis are shown below, as are illustrative and nonlimiting viral agents, such as Vidarabine and conjugates and their methods of synthesis.

### Geranylgeranyl (GG)-Alcohol/Campothecin Conjugate

[chemical structure]

### Carbonate Containing GG-Alcohol/Campothecin Conjugate

[chemical structure]

### Carbamate GG-Alcohol/5-FU Codrug or Carrier Conjugate

[chemical structure]

### Vidarabine Conjugate

[chemical structure]

Other antiviral drugs may be attached in similar fashion to the GG-alcohol or GG-acetone.

Illustrative and non-limiting examples of antibiotics useful in such compounds and certain nonlimiting points of attachment (shown by an “→”) of such antibiotics to GGA or a GGA derivative are shown below.

[chemical structure]

Illustrative and non-limiting examples of glaucoma drugs useful in such compounds and certain nonlimiting points of attachment (shown by an “→”) of such drugs to GGA or a GGA derivative are shown below.

[chemical structure]

The configuration of compounds can be determined by methods known to those skilled in the art such as chiroptical spectroscopy and nuclear magnetic resonance spectroscopy.

### Synthesis of GGA Derivatives

Certain methods for making GGA or certain GGA derivatives provided and/or utilized herein are described in PCT publication no. WO 2012/031028 and PCT application no. PCT/US2012/027147, each of which are incorporated herein by reference in its entirety. Other GGA derivatives can be prepared by appropriate substitution of reagents and starting materials, as will be well known to the skilled artisan upon reading this disclosure.

The reactions are preferably carried out in a suitable inert solvent that will be apparent to the skilled artisan upon reading this disclosure, for a sufficient period of time to ensure substantial completion of the reaction as observed by thin layer chromatography, ¹H-NMR, etc. If needed to speed up the reaction, the reaction mixture can be heated, as is well known to the skilled artisan. The final and the intermediate compounds are purified, if necessary, by various art known methods such as crystallization, precipitation, column chromatography, and the likes, as will be apparent to the skilled artisan upon reading this disclosure.

The compounds provided and/or utilized in this invention are synthesized, e.g., from a compound of formula (III-A):

[chemical structure]

wherein n, R¹-R⁵ and [symbol] are defined as in Formula (I) above, following various well known methods upon substitution of reactants and/or altering reaction conditions as will be apparent to the skilled artisan upon reading this disclosure. The compound of Formula (III-A) is itself prepared by methods well known to a skilled artisan, for example, and without limitation, those described in PCT Pat. App. Pub. No. WO 2012/031028 and PCT Pat. App. No. PCT/US2012/027147 (each supra). An illustrative and non-limiting method for synthesizing a compound of Formula (III-A), where n is 1, is schematically shown below.

[chemical structure]

Starting compound (iii), which is synthesized from compound (i) by adding isoprene derivatives as described here, is alkylated with a beta keto ester (iv), in the presence of a base such as an alkoxide, to provide the corresponding beta-ketoester (v). Compound (v) upon alkaline hydrolysis followed by decarboxylation provides ketone (vi). Keto compound (vi) is converted, following a Wittig Horner reaction with compound (vii), to the conjugated ester (viii). Compound (viii) is reduced, for example with LiAlH₄, to provide alcohol (ix).

As will be apparent to the skilled artisan, a compound of Formula (III), where n is 2, is synthesized by repeating the reaction sequence of alkylation with a beta-keto ester, hydrolysis, decarboxylation, Wittig-Horner olefination, and LiAlH₄ reduction.

Certain illustrative and non-limiting synthesis of compounds provided and/or utilized in this invention are schematically shown below. Compounds where Q¹ is —(C═S)— or —SO₂— are synthesized by substituting the carbonyl group of the reactants employed, as will be apparent to the skilled artisan.

R⁶ in the schemes below may also correspond to R³⁰ and R⁵¹ as defined herein. R⁷ in the schemes below may also correspond to R²⁶, R³¹ and R⁵² as defined herein. R⁸ in the schemes below may also correspond to R²⁷, R³² and R⁵³ as defined herein. R⁹ in the schemes below may also correspond to R²⁸, R³³ and R⁵⁴ as defined herein. R¹³ in the schemes below may also correspond to R⁵⁸ as defined herein. R¹⁴ in the schemes below may also correspond to R⁵⁹ as defined herein. R¹⁵ in the schemes below may also correspond to R⁶⁰ as defined herein. R¹⁸ in the schemes below may also correspond to R²⁴, R³⁴ and R⁶³ as defined herein. R¹⁹ in the schemes below may also correspond to R²⁵, R³⁵ and R⁶⁴ as defined herein. L is a leaving group as known to one of ordinary skill in the art.

[chemical structure]

As shown above, R^{E} is alkyl.

Compound (ix) with alcohol functionality is an intermediate useful for preparing the compounds provided and/or utilized in this invention. Compound (x), where L is an R^{s}SO₂— group is made by reacting compound (ix) with R^{s}SO₂Cl in the presence of a base. The transformation of compound (iii) to compound (x) illustrates methods of adding isoprene derivatives to a compound, which methods are suitable to make compound (iii) from compound (i). Intermediate (ix) containing various R¹-R⁵ substituents are prepared according to this scheme as exemplified herein below. The transformation of compound (iii) to compound (x) illustrates methods of adding isoprene derivatives to a compound, which methods are suitable to make compound (iii) from compound (i).

The intermediates prepared above are converted to the compounds provided and/or utilized in this invention as schematically illustrated below:

[chemical structure]

As used herein, for example, and without limitation, m is 0 or 1 and R¹-R⁵ are as defined herein, and are preferably alkyl, or more preferably methyl. Intermediate (ixa), prepared according to the scheme herein above, is converted to amino intermediate (ixb) via the corresponding bromide. Intermediates (ixa) and (ixb) are converted to the compounds provided and/or utilized in this invention by reacting with suitable isocyanates or carbamoyl chlorides, which are prepared by art known methods. The thiocarbamates and thioureas of this invention are prepared according to the methods described above and replacing the isocyanates or the carbamoyl chlorides with isothiocyanates (R¹⁸—N═C═S) or thiocarbamoyl chlorides (R¹⁸—NH—C(═S)Cl or R¹⁸R¹⁹N—C(═S)Cl). These and other compounds provided and/or utilized in this invention are also prepared by art known methods, which may require optional modifications as will be apparent to the skilled artisan upon reading this disclosure. Intermediates for synthesizing compounds provided and/or utilized in this invention containing various R¹-R⁵ substituents are illustrated in the examples section and/or are well known to the skilled artisan.

Certain GGA derivatives provided and/or utilized herein are synthesized as schematically shown below.

[chemical structure]

Certain compounds provided and/or utilized herein are obtained by reacting compound (x) with the anion Q(-), which can be generated by reacting the compound QH with a base. Suitable nonlimiting examples of bases include hydroxide, hydride, amides, alkoxides, and the like. Various compounds provided, and/or utilized in this invention, wherein the carbonyl group is converted to an imine, a hydrazone, an alkoxyimine, an enolcarbamate, a ketal, and the like, are prepared following well known methods.

Other methods for making the compounds provided and/or utilized in this invention are schematically illustrated below:

[chemical structure]

The metallation is performed, by reacting the ketone with a base such as dimsyl anion, a hindered amide base such as diisopropylamide, or hexamethyldisilazide, along with the corresponding metal cation, M. The amino carbonyl chloride or the isocyanate is prepared, for example, by reacting the amine (R¹⁴)₂NH with phosgene or an equivalent reagent well known to the skilled artisan.

[chemical structure]

The beta keto ester is hydrolyzed while ensuring that the reaction conditions do not lead to decarboxylation. The acid is activated with various acid activating agent well known to the skilled artisan such as carbonyl diimodazole, or O-Benzotriazole-N,N,N′,N′-tetramethyl-uronium-hexafluoro-phosphate (HBTU) and reacted with the amine.

[chemical structure]

Various other compounds provided and/or utilized in this invention are prepared from the compounds made in the scheme above based on art known methods.

[chemical structure]

As shown above, R^{E} is alkyl.

The intermediates prepared above are converted to the compounds provided and/or utilized in this invention as schematically illustrated below:

[chemical structure]

Compound (viii) is hydrolyzed to the carboxylic acid (x), which is then converted to the acid chloride (xi). Compound (xi) is reacted with a suitable nucleophile such as a hydrazide, a hydroxylamine, an amino alcohol, or an amino acid, and the intermediate dehydrated to provide a compound of Formula (IV). Alternatively, the allylic alcohol (ix) is oxidized to the aldehyde (xi), which is then reacted with a cyanohydrin or cyanotosylmethane to provide further compounds provided and/or utilized in this invention.

GGA derivatives provided and/or utilized in this invention can also be synthesized employing art known methods and those disclosed here by alkene-aryl, alkene-heteroaryl, or alkene-akene couplings such as Heck, Stille, or Suzuki coupling. Such methods can use (vi) to prepare intermediate (xii) that can undergo Heck, Stille, or Suzuki coupling under conditions well known to the skilled artisan to provide compounds provided and/or utilized in this invention.

[chemical structure]

Higher and lower isoprenyl homologs of intermediates (x), (xi), and (xii), which are prepared following the methods disclosed here, can be similarly employed to prepare other compounds provided and/or utilized in this invention.

Compounds provided and/or utilized in this invention are also prepared as shown below

[chemical structure]

L is a leaving group and Q₅ are as defined herein, Ar is a preferably an aryl group such as phenyl, the base employed is an alkoxide such as tertiarybutoxide, a hydride, or an alkyl lithium such as n-butyl lithium. Methods of carrying out the steps shown above are well known to the skilled artisan, as are conditions, reagents, solvents, and/or additives useful for performing the reactions and obtaining the compound of Formula (IV) in the desired stereochemistry.

Other methods for making the compounds provided and/or utilized in this invention are schematically illustrated below:

[chemical structure]

The metallation is performed, by reacting the ketone with a base such as dimsyl anion, a hindered amide base such as diisopropylamide, or hexamethyldisilazide, along with the corresponding metal cation, M. The amino carbonyl chloride or the isocyanate is prepared, for example, by reacting the amine R¹³R¹⁴NH with phosgene or an equivalent reagent well known to the skilled artisan.

[chemical structure]

The beta keto ester is hydrolyzed while ensuring that the reaction conditions do not lead to decarboxylation. The acid is activated with various acid activating agent well known to the skilled artisan such as carbonyl diimodazole, or O-Benzotriazole-N,N,N′,N′-tetramethyl-uronium-hexafluoro-phosphate (HBTU) and reacted with the amine. Certain other methods of preparing the conjugates are shown below.

[chemical structure]

As shown above, R is a memantine or a riluzole residue.
Polyprenyl amine-GGA derivatives can be prepared by reductive amination employing the appropriate polyprenyl aldehyde, a primary or secondary amine and a borohydride reducing agent, as is well known to the skilled artisan. The reaction can be carried out in THF or diethyl ether, optionally in presence of a protic acid, preferably a mild protic acid catalyst.

Illustrative and nonlimiting methods of making antibiotic and glaucoma drug conjugates of GGA and derivatives thereof are schematically shown below and/or can be adapted by the skilled artisan based on this disclosure. See, also, Expert Opinion on Therapeutic Patents, Prodrug strategies in nasal drug delivery, 2002, Vol. 12, No. 3, Pages 331-340.

### Ciprofloxacin Conjugate

[chemical structure]

### Betaxolol Conjugate

[chemical structure]

[chemical structure]

### Apraclonidine Conjugate

[chemical structure]

### Eye Drop Formulation

The compositions are formulated for eye delivery. Such formulations are well known in the art and can be modified based on this disclosure. As is well known, such formulations comprise water and one or more excipients such as preservatives, antioxidants, tonicity adjusting agents, and the likes. In some embodiments, the excipients further comprise, Polaxemers® and similar agents that can undergo a sol to gel transition upon delivery on the ocular surface. Alternatively, the compositions can be formulated for injection into the eye. Such are also well known.

Some embodiments provided herein describe a eye drop or ophthalmic formulation comprising a GGA derivative and an inert, non-eye irritating, non-toxic eye drop formulation. Such formulations are well known, and commonly referred to in, for example, the Physician's Desk Reference for Ophthalmology (1982 Edition, published by Medical Economics Company, Inc., Oridell, N.J.), wherein numerous sterile ophthalmologic ocular solutions are reported, e.g., see pp. 112-114, which are incorporated by reference.

Eye drop or ophthalmic formulations may include an excipient for introducing the GGA derivative into the eye of a subject. Non-limiting examples of such an excipient for eye drop or ophthalmic formulations include a vehicle, tonicity adjusting agent, surfactant, stabilizer or anti-oxidant, viscosity imparting agent, acidic substance, preservative, diluent, wetting agent, and a buffering agent.

Reference is made herein to medicaments in the form of eye drops. In some embodiments, eye drops include solutions, suspensions, gels, creams and ointments intended for ophthalmic use. In some embodiments, the eye drops are applied with an eye dropper.

Some embodiments provided herein describe an eye drop formulation, wherein the concentration of a GGA derivative is about 0.0001-about 10 wt %, about 0.1-about 5 wt %, about 0.1-about 3 wt %, about 0.05-about 3 wt %, about 0.05-about 2 wt %, about 0.05-about 1 wt %, about 0.5-about 10 wt %, about 0.5-about 5 wt %, about 0.5-about 4 wt %, about 0.5-about 3 wt %, about 0.5-about 2 wt %, about about 1 wt %, about 10%, about 7%, about 5%, about 4%, about 3.5%, about 3%, about 2.5%, about 2%, about 1.5%, about 1%, about 0.5%, about 0.1%, or about 0.05%. As is apparent and well known to the skilled artisan, the concentration of the active agent can be adjusted during and prior to the ocular delivery such that an effective amount is administered.

Some embodiments provided herein describe an eye drop formulation that comprises a vehicle. Examples of suitable vehicles for the eye drop formulation include but are not limited to purified water and vegetable oils (e.g., olive oil, castor oil, sesame oil, etc.).

Also provided herein in some embodiments is an eye drop formulation wherein the formulation further comprises one or more tonicity adjusting agents. In some embodiments, the tonicity adjusting agent is 0.5% to 2% of saline. In specific embodiments, the saline is a 0.9% w/v sodium chloride solution). Other non-limiting examples of tonicity adjusting agents include potassium chloride, buffer salts, dextrin, glycerin, propylene glycol and mannitol.

Some embodiments provided herein describe an eye drop formulation that optionally comprises a surfactant. In some embodiments, non-ionic surfactants aid in dispersing the active ingredient (e.g., a GGA derivative) in suspensions and improve solution clarity. Non-limiting examples of suitable surfactants include sorbitan ether esters of oleic acid (e.g., polysorbate80 or Tween 20 and 80), polyoxyethylene hydrogenated castor oil, cremophor, sodium alkylbenzene sulfonate, glycerol, lecithin, sucrose ester, polyoxyethylene-alkyl ether, polyoxyl stearate, polyoxyl 40 stearate, polymers of oxyethylated octyl phenol (tyloxapol) and polyoxyethylene polyoxypropylene glycol. In some embodiments, the eye drop formulation comprises polysorbate80, polyoxyethylene hydrogenated castor oil, lecithin or combinations thereof. In some embodiments, the amount of surfactant is 0.2-30 times of a GGA derivative, but preferably 0.3-10 times of a GGA derivative. In some embodiments, an eye drop formulation comprises about 0.1-10 wt % of polysorbate80, polyoxyethylene hydrogenated castor oil, or lecithin. In some embodiments, an eye drop formulation comprises about 0.1-10 wt %, about 0.1-7 wt %, about 0.1-5 wt %, about 0.1-4 wt %, about 0.1-3 wt %, about 0.1-2 wt %, about 0.1-15 wt %, about 1-10 wt %, about 2-10 wt %, about 2-8 wt %, about 2-5 wt %, about 5-10 wt %, about 5-15 wt % of surfactant (e.g., polysorbate80, polyoxyethylene hydrogenated castor oil, or lecithin).

Some embodiments provided herein describe an eye drop formulation that optionally comprises a stabilizer or anti-oxidant. In some embodiments, the stabilizer or anti-oxidant decreases the rate of decomposition of active ingredient (e.g., a GGA derivative). Non-limiting examples of stabilizers and anti-oxidants include sodium bisulfate, sodium metabisulfite, ascorbic acid, isoascorbic acid, acetyl cysteine, 8-hydroxyquinoline, and thiourea.

Also provided herein in some embodiments is an eye drop formulation wherein the formulation further comprises one or more viscosity imparting agents. In some embodiments, viscosity imparting agents increase the viscosity of ophthalmic solution and suspension. In some embodiments, viscosity imparting agents increase ocular contact time, thereby decreasing the drainage rate. In some embodiments, viscosity imparting agents increase mucoadhesion, ocular bioavailability and/or impart a lubricating effect. Examples of viscosity imparting agents include but are not limited to poly vinyl alcohol, polyvinylpyrrolidone, methylcellulose, hydroxylpropylniethylcellulose, hydroxyethylcellulose, and carbomers.

In some embodiments, an acidic substance is optionally added. An example of an acidic substance is dimyristoylphosphatidic acid. Furthermore, adding dipalmitoylphosphatidylcholine (DPPG) results in more easily being able to prepare a clear solution. In some embodiments, anti-oxidants such as tocopherols or EDTA are added.

In some embodiments, preservatives are added to the eye drop formulation. In some embodiments, preservatives are anti-microbial or anti-bacterial agents. Parabens such as methylparaben and propylparaben, alcohol derivatives such as chlorobutanol, phenethyl alcohol, and benzyl alcohol, and organic acids such as sodium dehydroacetate, sorbic acid, and sodium sorbate are examples of such preservatives. Other examples of suitable preservatives include but are not limited to benzalkonium chloride, benzethonium chloride, polyquaternium-1 (Polyquad), thimerosal, phenylmercuric nitrate, phenylmercuric acetate, chlorobutanol, benzyl alcohol, sorbic acid, methyl paraben, propyl paraben, chlorhexidine, disodium EDTA, phenyl ethyl alcohol, polyaminopropyl biguanide, cetrimonium chloride, and purite. In some embodiments, the amount of preservative ranges from about 0.004% to about 0.02% by weight of the eye drop formulation.

Commonly used wetting agents are well known, and again are mentioned in the previously referred to pages of the Physician's Desk Reference for Ophthalmology. One suitable one is Tween, and in particular, Tween 80. In some embodiments, the amount of wetting agent ranges from 0.01% to 0.10%.

In some embodiments, the diluent is an isotonic eye treatment carrier, buffered to a pH within the range of from about 4.0 to about 8.0 and containing a small but effective amount of a wetting agent and an anti-bacterial agent.

Some embodiments provided herein describe an eye drop formulation optionally comprising one or more buffering agents. In some embodiments, the eye drops are buffered to about pH 7.4. In certain embodiments, the buffered eye drops maintain stability for at least 2 years. In some embodiments, the pH for the formulation described herein is within the range generally acceptable for eye drop, preferably pH 4-8 or about pH 7. The preferred pH range is from about 6.8 to about 7.8. Examples of suitable buffering agents include but are not limited to borate buffers and phosphate buffers (e.g., sodium phosphate).

For the manufacture of eye drop, a surfactant is added to a GGA derivative and mixed, and purified water is then added to the mixture. An isotonic agent such as sodium chloride and glycerin, buffer such as sodium phosphate, a pH-controlling agent such as dilute hydrochloric acid and sodium hydroxide, an antiseptic such as disodium edetate, an antifungal agent such as potassium sorbate, an anti-oxidizing agent such as tocophenol etc., is optionally added.

Eye drops are tested for various physicochemical, in vitro, and in vivo properties. Clarity is measured and ophthalmic solutions should be free from foreign particles. Visual and fluorescent microscopic methods are used for checking the clarity. The presence of particulate matter is also determined. Light obscuration or microscopic methods are used for counting and or measuring the particle size. The light obscuration particle count test determines number of particles 50/mL (≧10 μm diameter) or 5/mL (≧25 μm diameter). The microscopic particle count test determines the number of particles 50/mL (≧10 μm diameter) or 5/mL (≧25 μm diameter) or 2/mL (≧50 μm).

Isotonicity of the formulation is tested. Isotonic solutions do not change shape (bulging or shrinkage) of blood cells. Any change in the shape of blood cells is compared with standard marketed formulation. pH meters are used to measure the pH of eye drops. Sedimentation time for particles in ophthalmic suspension is measured by visual and microscopical methods.

Ophthalmic suspensions are evaluated for resuspendability. The container is inverted at the rate of about 8-10 times in a minute, and the number of inversions required to completely re-suspend the settled particles is noted.

Drug content in ophthalmic formulation is evaluated by suitable analytical methods such as UV, HPLC.

Eye drops are tested for preservative effectiveness as per guidelines given in USP 30. The test recommends for screening the eye drops for the absence of E. coli, S. aureus, P. aeruginosa, C. albicans and A. niger.

Limulus amoebocyte lysate (LAL) test is used for determination of bacterial endotoxins. The test (pyrogen test) involves measuring the rise in temperature of rabbits following the intravenous injection of a test solution.

The formulation is also sterilized. Various sterilization methods are used to sterilize the eye drops described herein, including steam sterilization, dry heat sterilization, gas sterilization, sterilization by ionizing radiation, sterilization by filtration, and aseptic processing.

### Methods of Treatment

Some embodiments provided herein describe a method of treating an ocular neural disease. In some instances, the ocular neural diseases are characterized by neuroinflammation. Also provided herein in some embodiments is a method of treating visual disorders such as optic neuropathy, glaucoma, degeneration of optic nerves, age-related macular degeneration (AMD) and ophthalmoplegia. Any pharmaceutical formulation and/or compounds described above are useful in the methods described herein.

Provided herein, in some embodiments, are methods for using effective amounts of a GGA derivative or the, optionally with at least one pharmaceutically acceptable excipient for inhibiting ocular neural death and/or increasing neural activity. For example, and without limitation, methods provided here in describe impeding the progression of ocular neural diseases or injury using one or more GGA derivatives.

In one aspect, methods for increasing the axon growth of ocular neurons by contacting said neurons with the pharmaceutical compositions are provided herein. In some cases, ocular neural diseases result in an impairment of signaling between ocular neurons. In some cases, this impairment is due in part to a reduction in the growth of axonal projections. In some embodiments, contacting neurons with a GGA derivative enhances axonal growth. In some embodiments, a GGA derivative restores axonal grown in neurons afflicted with an ocular neural disease. In a related embodiment, the pre-contacted neurons exhibit a reduction in the axon growth ability.

One embodiment provided herein describes a method for inhibiting the cell death of ocular neurons susceptible to neuronal cell death, which method comprises contacting said neurons with the pharmaceutical compositions provided herein. Ocular neurons susceptible to neuronal cell death include those that have the characteristics of a neural disease and/or those that have undergone injury or toxic stress.

In another aspect, there are methods for increasing the ocular neurite growth of ocular neurons by contacting said neurons with the pharmaceutical compositions provided herein. The term “neurite” refers to both axons and dendrites. Ocular neural diseases can result in an impairment of signaling between ocular neurons. In some cases, this impairment is due in part to a reduction in the growth of axonal and/or dendritic projections. It is contemplated that contacting neurons with a GGA derivative will enhance ocular neurite growth. It is further contemplated that a GGA derivative will restore neurite grown in neurons afflicted with an ocular neural disease. In a related embodiment, the pre-contacted neurons exhibit a reduction in the neurite growth ability.

One embodiment of this invention is directed to a method for increasing the expression and/or release of one or more ocular neurotransmitters from an ocular neuron by contacting said neuron with the pharmaceutical compositions provided herein. It is contemplated that contacting ocular neurons with an effective amount of a GGA derivative will increase the expression level of one or more ocular neurotransmitters. It is also contemplated that contacting ocular neurons with a GGA derivative will increase the release of one or more ocular neurotransmitters from neurons. The release of one or more ocular neurotransmitters refers to the exocytotic process by which secretory vesicles containing one or more ocular neurotransmitters are fused to cell membrane, which directs the ocular neurotransmitters out of the neuron. It is contemplated that the increase in the expression and/or release of ocular neurotransmitters will lead to enhanced signaling in neurons, in which levels of expression or release of ocular neurotransmitters are otherwise reduced due to the disease. The increase in their expression and release can be measured by molecular techniques commonly known to one skilled in the art.

One embodiment of this invention is directed to a method for inducing synapse formation of an ocular neuron by contacting said neuron with the pharmaceutical compositions provided herein. A synapse is a junction between two neurons. Synapses are essential to neural function and permit transmission of signals from one neuron to the next. Thus, an increase in the neural synapses will lead to an increase in the signaling between two or more neurons. It is contemplated that contacting the neurons with an effective amount of a GGA derivative will increase synapse formation in an ocular neurons that otherwise experience reduced synapse formation as a result of neural disease.

Another embodiment of this invention is directed to a method for increasing electrical excitability of an ocular neuron by contacting said neuron with the pharmaceutical compositions provided herein. Electrical excitation is one mode of communication among two or more neurons. It is contemplated that contacting neurons with an effective amount of a GGA derivative will increase the electrical excitability of ocular neurons in which electrical excitability and other modes of neural communication are otherwise impaired due to neural disease. Electrical excitability can be measured by electrophysiological methods commonly known to one skilled in the art.

In another embodiment, this invention is directed to a method for inhibiting the death of ocular neurons due to formation of or further formation of pathogenic protein aggregates between, outside or inside neurons, wherein said method comprises contacting said neurons at risk of developing said pathogenic protein aggregates with the pharmaceutical compositions provided herein. In one embodiment of this invention, the pathogenic protein aggregates form between or outside of the neurons. In another embodiment of this invention, the pathogenic protein aggregates form inside said neurons. In one embodiment of this invention, the pathogenic protein aggregates are a result of toxic stress to the cell.

Another embodiment of the invention is directed to a method for protecting ocular neurons from pathogenic extracellular protein aggregates which method comprises contacting said neurons and/or said pathogenic protein aggregates with the pharmaceutical compositions provided herein. In one embodiment of this invention, contacting said neurons and/or said pathogenic protein aggregates with the pharmaceutical compositions provided herein. There are many assays known to one skilled in the art for measuring the protection of neurons either in cell culture or in a mammal.

In yet another embodiment of the invention is directed to a method for protecting ocular neurons from pathogenic intracellular protein aggregates which method comprises contacting said neurons with the pharmaceutical compositions provided herein.

One embodiment of the invention is directed to a method of modulating the activity of G proteins in ocular neurons which method comprises contacting said neurons with the pharmaceutical compositions provided herein. It is contemplated that contacting neurons with a GGA derivative will alter the sub-cellular localization, thus changing the activities of the G protein in the cell. In one embodiment of the invention, contacting neurons with a GGA derivative will enhance the activity of G proteins in ocular neurons. It is contemplated that contacting a GGA derivative with neurons will increase the expression level of G proteins. It is also contemplated that contacting a GGA derivative with optical neurons will enhance the activity of G proteins by changing their sub-cellular localization to the cell membranes where they must be to exert their biological activities.

One embodiment of the invention is directed to a method of modulating or enhancing the activity of G proteins in ocular neurons at risk of death which method comprises contacting said neurons with the pharmaceutical compositions provided herein.

One embodiment of the invention is directed to a method for inhibiting ocular neural death and increasing ocular neural activity in a mammal suffering from ocular neural diseases, wherein the etiology of said neural diseases comprises formation of protein aggregates which are pathogenic to ocular neurons, and which method comprises administering to said mammal the pharmaceutical compositions provided herein. This method is not intended to inhibit ocular neural death and increase ocular neural activity in ocular neural diseases in which the pathogenic protein aggregates are intranuclear or diseases in which the protein aggregation is related to SBMA.

In some embodiments, a pharmaceutical formulation described herein exerts cytoprotective effects on the eye and brain. (See, for example Ishii Y., et al., Invest Ophthalmol Vis Sci 2003; 44:198292; Tanito M, et al., J Neurosci 2005; 25:2396-404; Fujiki M, et al., J Neurotrauma 2006; 23:1164-78; Yasuda H, et al., and Brain Res 2005; 1032:176-82.

Some embodiments provided herein describe methods for treating eye-related diseases, disorders or conditions with a GGA derivative. Examples of eye-related or visual disorders include but are not limited to macular degeneration, retinitis pigmentosa, glaucoma, and/or retinal degeneration.

In some embodiments, a pharmaceutical formulation described herein comprising a GGA derivative is used for treating glaucoma. Glaucoma is a degenerative disease of the eye characterized by progressive optic nerve damage with selective loss of retinal ganglion cells. In some instances, apoptosis leads to retinal ganglion cell death in glaucoma. In some instances, the intraocular pressure remains elevated for prolonged time periods, the fibers of the optic nerve atrophy and/or the retina loses function.

Accordingly, provided herein is a method of inhibiting apoptosis-like cell death of retinal ganglion cells comprising administering to the retinal ganglion cell a pharmaceutical formulation comprising a GGA derivative. In some embodiments, a method is provided for enhancing the survival of retinal ganglion cells. In further or additional embodiments, a method is described protecting retinal ganglion cells from damage or cell death. Also provided herein in some embodiments is a method for inducing expressing of heat shock proteins (e.g., HSP72) in a retinal neuron. In some embodiments, a method of ameloriating glaucomatous damage to an eye comprises administration of a pharmaceutical formulation comprising a GGA derivative. In other embodiments, a method is provided for preventing axonal injury in an optic nerve, the method comprising administering to the eye a pharmaceutical formulation comprising a GGA derivative. Some embodiments provided herein describe a method of reducing elevated intraocular pressure in an eye comprising administering to the eye a pharmaceutical formulation comprising a GGA derivative. In specific embodiments, the pharmaceutical formulation is administered to the eye as a drop, spray or ointment.

In certain aspects, the methods described herein relate to administering a GGA derivative or the isomeric compounds or compositions thereof in vitro. In other aspects the administration is in vivo. In yet other aspects, the in vivo administration is to a mammal. Mammals include but are not limited to humans and common laboratory research animals such as, for example, mice, rats, dogs, pigs, cats, and rabbits.

Compounds, compositions and methods of the invention described herein include the disclosures found in international application No.: PCT/US2011/050071, filed on Aug. 31, 2011 and the international PCT application entitled “GERANYLGERANYLACETONE DERIVATIVES”, filed on Feb. 29, 2012, both of which are incorporated herein in its entirety by reference. All citations herein are incorporated herein by reference in their entirety.

Method of treating bacterial infections, viral infections, or cancers of the eye, brain, and spinal chord, and the nerves in the brain, eye, and the spinal chord are well known in the art and can be appropriately adapted for practicing the methods of this invention upon reading this disclosure by the skilled artisan.

### EXAMPLES

### Example 1

### Eye Drop Formulation of a GGA Derivative

Eye drops are prepared by dissolving a GGA derivative (1.0 g) in a phosphate buffer solution which is prepared by dissolving 0.8 g of sodium dihydrogen phosphate and 0.5 g of sodium chloride in purified water such that the final weight is 100 g. The pH is adjusted to 7.0 with sodium hydroxide.

### Example 2

### Eye Drop Formulation

Eye drops are prepared by dissolving a GGA derivative (1.0 g) in 1.0 g of dimethyl sulfoxide and adding the resulting solution to a boric acid solution prepared by dissolving 2.0 g of boric acid in purified water such that the final weight is 100 g. The pH is adjusted to 7.0 with sodium hydroxide.

### Example 3

### Eye Drop Formulation

| GGA derivative | 1.0 | g |
|---|---|---|
| Potassium sorbate | 0.1 | g |
| Polysorbate80 | 0.5 | g |
| Sodium chloride | 0.9 | g |
| Disodium edetate | 0.01 | g |
| Sodium hydroxide | as appropriate | |
| Dilute hydrochloric acid | as appropriate | |
| Total Volume | 100 | mL |

Polysorbate80 is added to a GGA derivative in sterile purified water. After mixing, potassium sorbate, sodium chloride, and disodium edetate in sterile purified water, water is added to the mixture and stirred. The pH is adjusted to 6.5 by adding sodium hydroxide in sterile purified water and dilute hydrochloric acid.

### Example 4

### Eye Drop Formulation

The eye drop formulation (in 100 mL) is prepared following similar methods described in Example 3.

| | |
|---|---|
| GGA derivative | 1.0 g |
| Potassium sorbate | 0.2 g |
| Polysorbate80 | 0.5 g |
| Sodium chloride | 0.81 g |
| Disodium edetate | 0.01 g |
| Sodium hydroxide | as appropriate |
| Dilute hydrochloric acid | as appropriate |

### Example 5

### Eye Drop Formulation

The eye drop formulation (in 100 mL) is prepared following similar methods described in Example 3.

| | |
|---|---|
| GGA derivative | 0.5 g |
| Potassium sorbate | 0.2 g |
| Polysorbate80 | 0.25 g |
| Sodium chloride | 0.81 g |
| Disodium edetate | 0.01 g |
| Sodium hydroxide | as appropriate |
| Dilute hydrochloric acid | as appropriate |

### Example 6

### Eye Drop Formulation

The eye drop formulation (in 100 mL) is prepared following similar methods described in Example 3.

| | |
|---|---|
| GGA derivative | 0.2 g |
| Potassium sorbate | 0.5 g |
| Polyoxyethylene hydrogenated castor oil | 2.0 g |
| Sodium chloride | 0.8 g |
| Disodium edetate | 0.01 g |
| Sodium hydroxide | as appropriate |
| Dilute hydrochloric acid | as appropriate |

### Example 7

### Eye Drop Formulation

The eye drop formulation (in 100 mL) is prepared following similar methods described in Example 3.

| | |
|---|---|
| GGA derivative | 5.0 g |
| Potassium sorbate | 1.0 g |
| Polyoxyethylene hydrogenated castor oil | 2.5 g |
| Sodium chloride | 0.8 g |
| Disodium edetate | 0.05 g |
| Sodium hydroxide | as appropriate |
| Dilute hydrochloric acid | as appropriate |

### Example 8

### Eye Drop Formulation

| GGA derivative | 100 | mg |
|---|---|---|
| Egg yolk lecithin | 50 | mg |
| DMPA (dimyristoylphosphatidic acid) | 10 | mg |
| Tween 80 | 50 | mg |
| Vitamin E | 1 | mg |
| Taurine | 60 | mg |
| Potassium sorbate | 20 | mg |
| 10 mM EDTA-2 Na | 0.2 | mL |
| Sorbitol | 9.6 | mg |
| Sodium hydroxide in water | as appropriate | |
| Sterile water | as appropriate | |
| Total volume | 10 | mL |

The eye drop in this invention is manufactured in the following fashion. After dissolving a GGA derivative, egg yolk lecithin (the phospholipid), and tocopherol acetate in a solvent mixture of chloroform and methanol, the solvent is distilled off using an evaporator, leaving a thin film of lipids. 5% glucose solution is added and shaken to suspend the lipids, then exposed to ultrasound, for example 15 minutes in a 40° C. ultrasonic bath. A synthetic surfactant, Tween 80 solution for example, is added, and then more 5% glucose solution is added to produce a clear GGA derivative-containing eye drop.

### Example 9

### Eye Drop Formulation

| GGA derivative | 100 | mg |
|---|---|---|
| Egg yolk lecithin | 35 | mg |
| DMPA | 7 | mg |
| Tween 80 | 50 | mg |
| Vitamin E | 1 | mg |
| Taurine | 60 | mg |
| Potassium sorbate | 20 | mg |
| 10 mM EDTA-2 Na | 0.2 | mL |
| Sorbitol | 9.6 | mg |
| Sodium hydroxide in water | as appropriate | |
| Sterile water | as appropriate | |
| Total volume | 10 | mL |

### Example 10

### Eye Drop Formulation

| GGA derivative | 100 | mg |
|---|---|---|
| Egg yolk lecithin | 15 | mg |
| DMPA | 3 | mg |
| Tween 80 | 50 | mg |
| Vitamin E | 1 | mg |
| Taurine | 60 | mg |
| Potassium sorbate | 20 | mg |
| 10 mM EDTA-2 Na | 0.2 | mL |
| Sorbitol | 9.6 | mg |
| Sodium hydroxide in water | as appropriate | |
| Sterile water | as appropriate | |
| Total volume | 10 | mL |

### Example 11

### Eye Drop Formulation

| GGA derivative | 100 | mg |
|---|---|---|
| Egg yolk lecithin | 0 | mg |
| DMPA | 0 | mg |
| Tween 80 | 50 | mg |
| Vitamin E | 1 | mg |
| Taurine | 60 | mg |
| Potassium sorbate | 20 | mg |
| 10 mM EDTA-2 Na | 0.2 | mL |
| Sorbitol | 9.6 | mg |
| Sodium hydroxide in water | as appropriate | |
| Sterile water | as appropriate | |
| Total volume | 10 | mL |

### Example 12

### Eye Drop Formulation

| GGA derivative | 100 | mg |
|---|---|---|
| Vitamin E | 1 | mg |
| Egg yolk lecithin | 50-100 | mg |
| DMPA | 0-12 | mg |
| Cholesterol | 0-16 | |
| Tween 80 | 50 | mg |
| Glycerin | 1-2 | mg |
| Potassium sorbate | 20 | mg |
| Britton-Robinson buffer | 0-1 | mL |
| 0.3M boric acid buffer pH 9 | 0-1 | mL |
| EDTA-2Na | 0-0.4 | mg |
| Sodium hydroxide in water | as appropriate | |
| Sterile water | as appropriate | |
| Total volume | 10 | mL |

### Example 13

### Permeability Study with Eye Drop Formulation

An ophthalmic solution is made up as follows: 1 mg/ml (0.1%) solution of GGA derivative in phosphate buffered saline (pH=7.4) is used for half of the experiments and 1 mg/ml (0.1%) solution of a GGA derivative in phosphate buffered acrylic acid suspension is used for the experiments on rabbit corneas.

Before each permeability experiment, rabbit cornea tissue specimens are thawed at room temperature in phosphate buffered saline (PBS, pH 7.4). Tissue disks are equilibrated for 10 minutes with PBS (pH 7.4) at 20° C. in both the donor and receiver compartments of the diffusion cells.

Following equilibration, the PBS is removed from the donor compartment and replaced with 1.0 mL of PBS, containing 1 mg/mL (0.1%) of a GGA derivative in PBS at pH 7.4 (w/v). PBS at 20° C. is pumped through the receiving chambers at a rate of 1.5 mL/h with a ISMATEC® 16 Channel High precision tubing pump and collected, by means of a ISCO Retriever IV fraction collector, at 2 h intervals for 24 h. The permeability studies are performed under sink conditions, i.e., at the completion of each run the concentration of a GGA derivative solution in the acceptor chamber never reaches 10% of that in the donor compartment. GGA derivative containing samples are collected in appropriate sampling tubes of the fraction collector. Samples are analyzed by HPLC with UV detection. The collected fractions are analyzed directly after completion of the respective experiment for GGA derivative content.

Calculation of Flux Values: Flux (J) values across membranes are calculated by means of the relationship J=Q/A×t (ng×cm-²×min^{˜1}) where Q indicates quantity of substance crossing membrane (in ng); A, membrane area exposed (in cm²); and t, time of exposure (in minutes).

Steady State Kinetics: when no statistically significant differences (p<0.05; analysis of variance and Duncan's multiple range test) between flux values are obtained over at least two consecutive time intervals, a steady state (equilibrium kinetics) is assumed to have been reached for a particular corneal specimen.

### Example 14

### Eye Drop Formulation and In Vivo Study

Eye drops are made by dissolving sufficient quantity of GGA derivative in distilled water to give 0.1%, 0.5%, 0.75%, and 2.0% solutions of a GGA derivative. Two drops are administered to the eye of normal and ocular induced hypertensive rabbits. The intraocular pressure of both the normal and ocular induced hypertensive rabbits is measured at intervals over a 6-hour period.

### Example 15

### Ocular Irritation Test

Rabbits are used as experimental animals (Draize test) for the measurement of redness, swelling, discharge, ulceration, hemorrhaging, cloudiness, or blindness in the tested eye. Confocal laser scanning ophthalmoscopy (CLSO) combined with corneal flourescein staining are also used.

### Example 16

### Rat Ocular Pharmacokinetics and Pharmacodynamics Study GGA Derivatives

Objective:

The objective of this study is to establish initial pharmacokinetic (PK) and pharmacodynamic (PD) data for an eye drop formulation containing a GGA derivative. The pharmacokinetics of a GGA derivative is determined and compared with vehicle controls, e.g., at different time points. One eye per rat is treated with a GGA derivative and one eye per rat is dosed with vehicle control according to the schedule shown in Table 1.

TABLE 1
Dosing Schedule for the PK study

| | | Treatment | | | | Time |
|---|---|---|---|---|---|---|
| Group | # of | Dose Level | Dose Level | Dose | Dosing | of eye |
| # | Rats | Left Eye | Right eye | Volume | times | harvest |
| 1a | 3M | GGA | 0 mg/eye | 5 μL | 0 h, 1 h, | 4 h |
| | | derivative | | | 2 h, 3 h | |
| | | 0.25 mg/eye | | | | |
| 2a | 3M | GGA | 0 mg/eye | 5 μL | 0 h, 1 h, | |
| | | derivative | | | 2 h, 3 h, | 8 h |
| | | 0.25 mg/eye | | | 4 h, 5 h, | |
| | | | | | 6 h, 7 h | |

HSP70 upregulation is analyzed by ELISA. One eye per rat is treated with the GGA derivative and one eye per rat is dosed with vehicle control according to the schedule shown in Table 2.

TABLE 2
Dosing Schedule for the HSP70 analysis

| | | Treatment | | | | Time |
|---|---|---|---|---|---|---|
| Group | # of | Dose Level | Dose Level | Dose | Dosing | of eye |
| # | Rats | Left Eye | Right eye | Volume | times | harvest |
| 1b | 4M | GGA | 0 mg/eye | 5 μL | 0 h, 1 h, | 4 h |
| | | derivative | | | 2 h, 3 h | |
| | | 0.25 mg/eye | | | | |
| 2b | 4M | GGA | 0 mg/eye | 5 μL | 0 h, 1 h, | 8 h |
| | | derivative | | | 2 h, 3 h, | |
| | | 0.25 mg/eye | | | 4 h, 5 h, | |
| | | | | | 6 h, 7 h | |
| 5b | 2M | Vehicle Ctrl | Vehicle | 5 μL | 0 h, 1 h, | 8 h |
| | | 0 mg/eye | Ctrl | | 2 h, 3 h, | |
| | | | 0 mg/eye | | 4 h, 5 h, | |
| | | | | | 6 h, 7 h | |

### Dose Administration:

| | |
|---|---|
| Route: | topical eye drop formulation |
| Frequency: | 4 or 8 doses, every 1 hour |
| Dose Administration: | under isofluorane anesthesia (2.5%) |
| Dose Volume: | 5 μL in each eye |

### Formulation for GGA Derivative:

0.005-20% GGA derivative
2.5% Hydrogenated castor oil
1% Potassium sorbate

### 0.8% NaCl

### 0.05% Disodium Edate

### In H₂O

pH 6.5

### Vehicle Control:

2.5% Hydrogenated castor oil
1% Potassium sorbate

### 0.8% NaCl

### 0.05% Disodium Edate

### In H₂O

pH 6.5

### Test Subjects:

| | |
|---|---|
| Species: | Rat |
| Strain: | Sprague-Dawley |
| Supplier: | Harlan |
| Sex: | Male |
| Weight at Initiation: | 200 to 220 g |
| Number of Animals: | 12 for Cohort 1, 26 for Cohort 2 |

It is contemplated that eye drop formulations of GGA derivatives of this invention at various concentrations, such as 0.005-5% can have the appearance similar or substantially similar to GGA formulations illustrated in FIG. 1.

### Example 17

### 1. HSP70 Induction after in Eyes by Eye Drops

Male Sprague-Dawley rats are administered an eye drop formulation containing a GGA derivative. Eye drops are applied every hour either for 4 hours or for 8 hours. Animals are euthanized 4 hours, 8 hours or 24 hours after the first dosing, and the eye balls collected on ice. Eyes are homogenized with a polytron homogenizer in a standard lysis buffer containing proteinase inhibitors. HSP70 is quantified by a commercially available ELISA kit and normalized by total protein concentration in the sample.

It is contemplated that administration of an eye drop formulation of as low as 0.05% of a GGA derivative can result in increased HSP70 expression in the eyeballs treated with that GGA derivative. [* The HSP 70 related figure was deleted because it showed GGA based data. HSP70 upregulation in the eye by GGA has been disclosed in prio filed application(s)]

### 2. PK after Administering Eye Drops

Male Sprague-Dawley rats are administered an eye drop formulation containing 1-10% GGA derivative. Eye drops are applied every hour either for 4 hours or for 8 hours. Animals are euthanized 4 hours and 8 hours after the first dosing, and the eye balls collected on dry ice. Eyes are homogenized with a polytron homogenizer in ethanol. GGA is quantified in the eye ball lysates by liquid chromatography-tandem mass spectroscopy.

### Example 18

### 1. PK Studies

Single dose of 0.005-20% GGA derivative is administered by eye drop to rat eye balls (both eyes). 4-5 time points including time 0 are taken, as is base line data. AUC (eye ball) is calculated. A percentage of an input delivered to eye balls is calculated.

### 2. HSP70 Inductions

Single dose of 0.005-20% GGA derivative is administered by eye drop to rat eye ball (both eyes). Eye balls are extracted at 2-3 time points. It is contemplated that HSP70 inductions in eye balls may be seen at different time points. Vehicle only controls using different animals are used. HSP70 induction in tissues dosed with GGA derivative or vehicle is determined.

### Example 19

### Parenteral Administration of GGA Derivative Through the Ocular Surface of a Patient

It is contemplated that a jetting device such as that described, e.g., and without limitation, in U.S. Pat. No. 7,563,244 can be used to administer an effective amount of a GGA derivative into the eye of a patient through the ocular surface of the patient. For example, a GGA derivative formulation can be added to a jetting device that dispenses the formulation into the eye by ejecting it as a vapor or as droplets towards the ocular surface of the patient, whereby the pharmaceutical formulation penetrates the ocular surface and delivers the GGA derivative into the eye of a patient.

### Example 21

Results of ocular, retinal delivery of GGA by eye drop compared to systemic delivery is determined: The (AUC(eye drop)/dose(eye drop))/(AUC(PO)/dose(PO)) is measured to determine the efficacy of GGA derivative delivery into the eye and/or the retina.

### Example 20

### Relative Bioavailability of a GGA Derivative in the Eyeball and Retina Following Ocular or Oral Administration

Rats are dosed once either with a 5% ocular topical emulsion or an oral suspension of a GGA derivative according to the experimental design in as tabulated below. Animals are sacrificed and eye balls harvested at 1 h, 2 h, and 4 h post-dose, respectively. From each animal, one eyeball is submitted for bioanalysis of a GGA derivative. From the second eye ball of each animal, the retina is dissected and submitted for bioanalysis.

### Experimental Design

| | | Treatment | | | | Time |
|---|---|---|---|---|---|---|
| Group | # of | Dose Level | Dose Level | Dose | Dosing | of eye |
| # | Rats | Left Eye | Right eye | Volume | times | harvest |
| 1 | 3M | GGA | GGA | 5 μL | 0 h | 1 h |
| | | derivative | derivative | | | |
| | | 250 ug/eye | 250 ug/eye | | | |
| 2 | 3M | GGA | GGA | 5 μL | 0 h | 2 h |
| | | derivative | derivative | | | |
| | | 250 ug/eye | 250 ug/eye | | | |
| 3 | 3M | GGA | GGA | 5 μL | 0 h | 4 h |
| | | derivative | derivative | | | |
| | | 250 ug/eye | 250 ug/eye | | | |

| | | | | |
|---|---|---|---|---|
| | | Dose Level for Oral Gavage | | |
| 7 | 3M | GGA derivative about | 0 h | 1 h |
| | | 180 mg/kg PO | | |
| 8 | 3M | GGA derivative about | 0 h | 2 h |
| | | 180 mg/kg PO | | |
| 9 | 3M | GGA derivative about | 0 h | 4 h |
| | | 180 mg/kg PO | | |

The GGA derivative-concentrations measured 1 h, 2 h, and 4 h after an ocular or oral single dose of the GGA derivative. It is contemplated that topical ocular administration of a 0.005-20% eye drop formulation of a GGA derivative results in substantially higher exposure in the retina and the eye ball than following a single oral dose of about 180 mg/kg of that GGA derivative.

Based on dose-adjusted AUC, topical ocular administration is 350 to 3700-times more efficient in delivering of CNS-101 or CNS-102 to the eye ball or retina than oral administration.

All abbreviations for scientific terms used herein have their ordinary scientific meaning as known to the skilled artisan.

1. A method for inhibiting optic nerve damage in a patient at risk of such damage which method comprises applying a therapeutically effective amount of a composition comprising 0.0005-20 wt % of a GGA derivative to or into an ocular surface of said patient in an amount sufficient to increase intraocular levels of HSP 70, thereby inhibiting the optic nerve damage.

2. A method of increasing HSP70 levels in ocular tissue comprising administering topically on the ocular surface an effective amount of a GGA derivative.

3. The method of claim 2, wherein the GGA derivative is administered as a trans isomer free of the cis isomer or as a mixture of cis and trans isomers.

4. The method of claim 1, further comprising providing an effective intraocular concentration of the GGA derivative.

5. The method of claim 1, wherein the composition comprises 0.1 wt % to 10 wt % of the GGA derivative.

6. The method of claim 1, wherein the composition comprises 3 wt % to 6 wt % of the GGA derivative.

7. The method of claim 1, wherein the GGA or the derivative thereof is a mixture of cis and trans-isomers.

8. The method of claim 1, wherein the intraocular levels of HSP 70 are increased by at least 10%.

9. The method of claim 1, wherein the optic nerve damage derives from or is related to glaucoma, macular degeneration, exposure to UV light, trauma, stroke, optic neuritis, ischemia, infection, compression from a tumor, compression from an aneurysm or Leber's hereditary optic neuropathy.

10. A pharmaceutical composition suitable for parenteral administration to a patient, wherein the pharmaceutical composition comprises a GGA derivative and at least one excipient for introducing the GGA derivative into the eye of a subject.

11. The pharmaceutical composition of claim 10, suitable for parenteral administration through the ocular surface of a patient via a jetting device.

12. The pharmaceutical composition of any one of claim 10, wherein the excipient comprises a tonicity adjustment agent.

13. The topical ocular composition of claim 12, wherein the tonicity adjusting agent is isotonic.

14. The topical ocular composition of claim 12, wherein the tonicity adjusting agent is saline, dextrose, glycerin, aqueous potassium chloride, buffer salts, propylene glycol, or mannitol.

15. The topical ocular composition of claim 12, wherein the tonicity adjusting agent is saline.

16. The topical ocular composition of claim 12 in the form of a topical eye drop.

17. The topical ocular composition of claim 12, wherein the composition further comprises one or more of a surfactant, an anti-bacterial agent, a pH buffering agent, an antioxidant agent, a preservative agent, a viscosity imparting agent or a combination thereof.

18. The topical ocular composition of claim 12 for use in the manufacture of a medicament for treatment of an ocular or visual disorder.

19. The topical ocular composition of claim 18, wherein the ocular or visual disorder is a neural disorder.

20. The topical ocular composition of claim 19, wherein the neural disorder is glaucoma, optic nerve degeneration or age-related macular degeneration.

21. A physiological supplement or medicament for ophthalmic use, in the form of eye drops, comprising a GGA derivative in a range of about 0.0005%-20%.

22. A formulation for treatment of an ocular neural disease, disorder or condition, comprising a GGA derivative, and at least one carrier material for introducing a GGA derivative into the eye of a subject suffering from the neural disease, disorder or condition.

23. The formulation of claim 22, further comprising one or more of a surfactant, an anti-bacterial agent, a pH buffering agent, an antioxidant agent, a preservative agent, or a combination thereof.

24. The formulation of claim 22, wherein said carrier material comprises an ocular/ophthalmic carrier.

25. The formulation of claim 22, wherein the neural disease, disorder, or condition is glaucoma, optic nerve degeneration or age-related macular degeneration.

26. A method of treating glaucoma, the method comprising administering to a subject in need thereof a pharmaceutical formulation comprising a GGA derivative.

27. The method of either of claim 26, wherein the formulation further comprises one or more of a tonicity adjusting agent, a surfactant, an anti-bacterial agent, a pH buffering agent, an antioxidant agent, a preservative agent, a viscosity imparting agent or a combination thereof.

28. The method of claim 26, wherein the formulation comprises 0.1-5%.

29. The method of claim 26, wherein the formulation is administered to the eye of the subject.

30. A method of inhibiting apoptosis of a retinal ganglion cell, the method comprising administration a pharmaceutical formulation of a GGA derivative to the cell.

31. The method of claim 30, wherein the pharmaceutical formulation further comprises an ocular/ophthalmic carrier.

32. The method of claim 30, wherein the retinal ganglion cell is present in an individual.

33. The method claim 30, wherein the individual is in need of glaucoma therapy.

34. The method of claim 30, wherein the pharmaceutical formulation is administered to the subject by an eye drop.

35. An eye drop for the treatment of an ocular neural disease, disorder or condition through topical application of said eye drop to the eye of a subject suffering from said disease, disorder or condition, comprising a therapeutically effective amount of a GGA derivative.

36. A method of delivering a GGA derivative into a retina of a subject, the method comprising ocular administration to the subject of the GGA derivative.

37. A method of treating a retinal disease in a subject, the method comprising administering topically on an ocular surface of the subject an effective amount of a GGA derivative.

38. A method of inhibiting a retinal optical nerve damage in a subject, the method comprising ocular administration to the subject of an effective amount of a GGA derivative.

39. The method of claim 36, wherein the GGA derivative is delivered into the eye or into the retina of the subject 50-10,000 times or 500-5,000 times more efficiently by ocular delivery compared to systemic such as oral delivery.
